(19) United States
(12) Patent Application Publication
(10) Pub. No.: US 2010/0047277 A1
(43) Pub. Date: Feb. 25, 2010

(54) VIROSOMES, METHODS OF PREPARATION, AND IMMUNOGENIC COMPOSITIONS

(76) Inventors: Richard W. Compans, Atlanta, GA (US); Chinglai Yang, Decatur, GA (US); Qizhi Yao, Houston, TX (US); Sang-moo Kang, Norcross, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/373,595

(22) PCT Filed: Jul. 12, 2007
(86) PCT No.: PCT/US07/73342

§ 371 Date: Aug. 14, 2009

Related U.S. Application Data
(60) Provisional application No. 60/830,762, filed on Jul. 13, 2006.

(51) Int. Cl. A61K 39/12; A61K 39/21; A61K 39/145; A61K 39/155; A61K 39/215; A61K 39/205; A61K 39/245; A61K 39/25; C12Q 1/70; C12N 15/00; A61P 31/12

(52) U.S. Cl. 424/208.1; 424/204.1; 424/207.1; 424/209.1; 424/211.1; 424/218.1; 424/221.1; 424/224.1; 424/229.1; 424/230.1; 435/5; 435/455

(57) ABSTRACT

Briefly described, virosomes, methods of preparing virosomes, immunogenic compositions that include virosomes, and methods of eliciting an immune response using immunogenic compositions that include virosomes are described herein. A virosome can include at least one viral surface envelope glycoprotein expressed on the surface of the virosome. The virosome can also optionally include at least one adjuvant molecule expressed on the surface of the virosome.

### CROSS-REFERENCE TO RELATED APPLICATION

This application is a continuation-in-part of U.S. application Ser. No. 11/397,830 filed Apr. 4, 2006 and claims the benefit of United States Provisional Patent Application No. 60/830,762 filed Jul. 13, 2006, the complete disclosures of which are incorporated herein by reference in there entirety.

### STATEMENT REGARDING FEDERALLY SPONSORED RESEARCH OR DEVELOPMENT

This invention was made with government support under grant numbers 1R21 AI53514, 1R21 AI44409, and 5 U01 AI056550 awarded by the National Institute of Health. The government has certain rights in the invention.

### TECHNICAL FIELD

The present disclosure is generally related to virosomes, particularly chimeric virosomes, methods of preparing virosomes, immunogenic compositions that include virosomes, and methods of eliciting an immune response using immunogenic compositions that include virosomes.

### BACKGROUND

Virosomes somewhat resemble mature virions, but they do not contain viral genomic material (e.g., viral genomic RNA or DNA). Therefore, virosomes are nonreplicative in nature, which make them safe for administration in the form of an immunogenic composition (e.g., vaccine). In addition, virosomes are lipid vesicles that contain envelope glycoproteins on the surface of the vesicle. Moreover, since virosomes have a surface structure that resembles intact virions, virosomes may be more effective in inducing neutralizing antibodies to the envelope glycoprotein than soluble viral envelope antigens. Further, virosomes are relatively simple to produce and can be administered repeatedly to vaccinated hosts, unlike many recombinant vaccine approaches.

Therefore, virosomes can be used to overcome problems encountered in previous attempts to create vaccines for various viruses such as human immunodeficiency virus (HIV), Ebola virus, severe acute respiratory syndrome (SARS), coronavirus, influenza virus, parainfluenza virus, and Rift Valley Fever virus (RVFV).

### SUMMARY

Briefly described, embodiments of the present disclosure include novel types of virosomes, in particular RVFV virosomes, Ebola virosomes, chimeric virosomes, methods of preparing virosomes, immunogenic compositions that include virosomes, and methods of eliciting an immune response using immunogenic compositions that include virosomes. “Virosomes” as used herein refers to membrane-enclosed vesicles containing viral glycoproteins on the surface of the vesicle.

One exemplary embodiment of a novel type of virosome includes at least one chimeric viral surface envelope glycoprotein expressed on the surface of the virosome. Another exemplary embodiment of a novel type of virosome includes a chimeric virosome having at least two viral surface envelope glycoproteins expressed on the surface of the virosome, where at least one of the viral surface envelope glycoproteins is from a different virus than at least one other viral surface envelope glycoprotein. Another exemplary embodiment of a virosome includes at least one viral surface envelope glycoprotein expressed on the surface of the virosome and at least one adjuvant molecule expressed on the surface of the virosome.

Another representative embodiment of the present disclosure includes an immunogenic composition. The immunogenic composition includes a pharmacologically acceptable carrier (e.g., an adjuvant composition, such as, but not limited to, Ribi, QS21, etc.) and at least one of the virosomes described above. Further, another representative embodiment of the present disclosure includes a method of generating an immunological response in a host by administering an effective amount of one or more of the immunogenic compositions described above to the host. Furthermore, another representative embodiment of the present disclosure includes a method of treating a condition by administering to a host in need of treatment an effective amount of one or more of the compositions described above.

Still another representative embodiment of the present disclosure includes methods of determining exposure of a host to a virus. An exemplary method, among others, includes contacting a biological fluid of the host with one or more of the virosomes discussed above, where at least one of the viral surface envelope glycoprotiens (or a portion thereof) of the virosome is of the same virus type to which exposure is being determined, under conditions permissive for binding of antibodies in the biological fluid with the virosome. The binding of antibodies within the biological fluid with the virosome is detected, whereby exposure of the host to the virus is determined by the detection of antibodies bound to the virosome.

Still another representative embodiment of the present disclosure includes methods of making virosomes. Virosomes can be produced in a number of ways, including, for instance, reconstitution of viral glycoproteins (purified) with liposomes. An exemplary embodiment of a novel method of making virosomes according to the present disclosure includes directly expressing membrane-bound viral glycoproteins in cells, whereby the cells produce the virosomes. Exemplary embodiments of such methods are described in greater detail below. An exemplary method, among others, includes the steps of: providing a viral surface envelope surface glycoprotein expression vector, optionally providing a membrane-anchored adjuvant molecule expression vector, introducing into a cell the viral surface envelope surface glycoprotein expression vector, and the optional adjuvant molecule expression vector, and allowing for expression of the viral surface envelope surface glycoprotein and the optional adjuvant molecule, whereby the virosome is formed by the cells.

Another embodiment of a method of making virosomes includes the steps of: providing one or more expression vectors including polynucleotide sequences encoding for at least one viral surface envelope glycoprotein and at least one adjuvant molecule; introducing the one or more expression vectors into a host cell; and expressing the at least one viral surface envelope glycoprotein and the at least one adjuvant molecule, whereby the virosome is formed by the cell.

Other systems, methods, features, and advantages of the present disclosure will be or will become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates a representative virosome (virosome).

FIG. 2 illustrates some representative structural changes that can be made to a representative viral surface envelope glycoprotein.

FIG. 3 illustrates some representative structural changes that can be made to another representative viral surface envelope glycoprotein.

FIG. 4 illustrates a schematic diagram of modified HIV envelope glycoproteins having the transmembrane and/or cytoplasmic domains of the HIV envelope glycoprotein replaced with the transmembrane and/or cytoplasmic domains of the MMTV envelope glycoprotein (MMTV), the baculovirus glycoprotein Gp64 (Gp64), the Lassa Fever virus glycoprotein (LFV), or the influenza surface glycoprotein HA (HA).

FIG. 5 illustrates a schematic diagram of modified HIV envelope glycoproteins having the transmembrane and/or cytoplasmic domains of the HIV envelope glycoprotein replaced with the transmembrane and/or cytoplasmic domains of the envelope glycoprotein of MMTV or LFV. FIG. 5 also illustrates a schematic diagram of a modified HIV envelope glycoprotein having the signal peptide domain of the HIV envelope glycoprotein replaced with the signal peptide domain of the mellitin protein.

FIG. 6A illustrates a Western blot analysis of RVFV virosomes and virus like particles (VLPs). The virosomes/VLPs were analyzed by SDS-PAGE (5 ug per lane) followed by Western blot using antibodies against the SIV Gag protein or monoclonal antibodies against the RVFV GN protein. Lanes 1, GP virosome; 2, GP-Gag VLP; 3, SIV-Gag VLP; M, molecular weight marker. FIG. 6B presents graphs illustrating the amount of RVFV glycoproteins in virosomes/VLPs. The virosomes and VLPs were lysed in lysis buffer and then coated onto a microtiter plate (1 ug per well). The levels of RVFV glycoproteins in virosomes/VLPs were compared by ELISA using mouse sera against RVFV as the primary antibody and HRP-conjugated rabbit-anti-mouse IgG as the secondary antibody. The control wells were coated with 1 ug of SIV Gag VLPs. A standard curve was constructed by coating the microtiter plate with serial dilutions of purified RVFV GN-histag proteins mixed with 1 ug SIV Gag VLPs for calculating the amount of RVFV glycoproteins in the virosomes and VLPs.

FIG. 7 illustrates an EM examination of GP virosomes and RVFV GP-SIV Gag VLPs. FIGS. 7A and B illustrate RVFV GP virosomes (A) and RVFV GP-SIV Gag VLPs (B) absorbed on a carbon grid and stained with 1% uranyl acetate followed by examination under a transmission electron microscope.

FIGS. 8A-8B illustrate the co-localization of RVFV GP with the SIV Gag proteins in sucrose gradients. The purified RVFV GP virosomes and RVFV GP-Gag VLPs (100 ug virosomes/VLPs dissolved in 500 ul PBS) were overlaid onto the top of a discontinuous sucrose gradient (2 ml of 10%, 20%, 30%, 40%, 50%, and 60% sucrose) and centrifuged in a SW41 rotor at 30000 rpm at 4° C. for 2 hr. After centrifugation, the six sucrose gradient layers were carefully collected and the proteins in each fraction were pelleted by centrifugation at 35000 rpm for 1 hr, and the pellets were resuspended in 60 ul of PBS. FIG. 8A illustrates detection of RVFV GN and SIV Gag proteins by Western blot using antibodies against RVFV GN or SIV Gag. 10 ul of each fraction (1 to 6 from top to bottom) were loaded per lane as indicated. Lanes M, molecular weight marker. FIG. 8B illustrates a comparison of the amounts of RVFV GP and SIV Gag proteins in each fraction by ELISA. A Microtiter plate was coated with 10 ul of each fraction per well and the levels of RVFV GP and SIV Gag proteins in each fraction were compared by ELISA using antibodies against RVFV or SIV Gag respectively. The control wells (fractions 7) were coated with 10 ug of Ebola VP40 VLPs.

FIGS. 9A-9C illustrate the analysis of antibody response induced by immunization with RVFV GP virosomes or RVFV VLPs as described in Example 2. Mice were immunized with GP virosomes, GP-Gag VLPs, or SIV Gag VLPs (Control) as described in the text. Blood samples were collected two weeks after each immunization, heat inactivated, and then analyzed for antibodies to RVFV. For FIG. 9A, microtiter plates were coated with purified RVFV GN-Histag proteins (0.4 ug per well) and the levels of antibodies against GN were determined by ELISA following standard procedures. A standard curve was generated by coating the wells with serial dilutions of purified mouse IgG with known concentrations for calculating the concentrations of GN-specific antibodies in serum samples. Numbers 1, 2, 3, and 4 indicate the serum samples collected after first, second, and third immunizations and 10 weeks after the third immunization, respectively. Error bars indicated standard variations in each group. For FIG. 9B, MP12 virus (an attenuated RVFV) was neutralized by sera from immunized mice after each immunization. MP12 (100 pfu) was incubated with serum samples (pooled for each group after each immunization) at 1:40 dilution in DMEM at 37° C. for 1 hr. The virus-serum mixtures were then added to VERO E6 cells seeded in a 12-well plate and titered by a plaque assay. MP12 virus incubated with DMEM was used as controls. The neutralizing activity of serum samples was calculated as the percentage of plaque number reduction as compared to the control wells (Percentage of neutralization). FIG. 9C illustrates the neutralization of MP12 by individual serum samples from each group after the third immunization.

FIG. 10A is an illustration of a schematic diagram of GN-MuC and GC-MuC chimeric proteins in which the cytoplasmic tails of the GN and GC proteins were replaced with the MuLV Env cytoplasmic tail. FIGS. 10B and 10C illustrate the expression and release of MuLV Gag and chimeric GN-MuC and GC-MuC proteins in Sf9 cells by recombinant baculoviruses by Western Blot and ELISA, respectively. For FIG. 10B, lane 1 shows GN-MuC+GC-MuC+MuLV-Gag (VLPs); lane 2 shows GN-MuC+GC-MuC (virosomes); lane 3 shows MuLV-Gag (control); and lane M shows the molecular weight marker.

FIG. 11A illustrates the budding of Ebola VLPs from insect cells. Sf9 cells were infected with rBV-VP40. At 24 hr post infection, the cells were fixed with 2% glutaraldehyde and then with 2% osmium tetroxide. Thin-section samples were prepared and stained with 1% uranyl acetate followed by examination under a transmission electron microscope. FIG. 11B illustrates negative staining of Ebola VLPs, and FIG. 11C illustrates the negative staining of pleiomorphic GP virosomes. Sf9 cells were infected with rBV-VP40 along with rBV-GP (B) or rBV-GP alone (C). At 48 hr post infection, medium was collected and VLPs or GP virosomes in medium were purified by centrifugation through a 10-50% sucrose gradient, and the visible bands containing VLPs or GP virosomes were collected, concentrated by centrifugation, and resuspended in PBS at a protein concentration of 1 ug/ul. The samples were stained with 1% uranyl acetate followed by examination under a transmission electron microscope. Arrows indicate elongated VLPs in A and B.

FIG. 12A illustrates western blot analysis of Ebola virosome and VLP preparations. Sf9 cells were infected with rBV-SIV-Gag (control VLP, lane 1); rBV-VP40 (lane 2); rBV-GP (lane 3); or rBV-VP40 and rBV-GP (lane 4). The virosome and VLP preparations were resuspended in PBS at the concentration of 1 ug/ul and 5 ug of virosome/VLP preparations were mixed with reducing protein sample buffer, heated at 95° C. for 5 min, and then subjected to SDS-PAGE followed by Western blot analysis using a mixture of monoclonal antibodies against Ebola VP40 and GP proteins. FIG. 12B illustrates mobility of the GP protein in SDS-PAGE in the presence or absence of the reducing reagent β-mercaptoethanol. Ebola VP40-GP VLPs (5 ug) were mixed with reducing (with β-mercaptoethanol, lane 3) or non-reducing (without β-mercaptoethanol, lane 2) protein sample buffers, heated at 95° C. for 5 min, and then subjected to SDS-PAGE followed by Western blot analysis using a monoclonal antibody against the Ebola GP protein. SIV-Gag VLPs mixed with non-reducing protein sample buffer were used as controls (lane 1). FIG. 12C illustrates Coomassie blue staining of Ebola virosome and VLP preparations. 5 ug of virosome/VLP preparations were mixed with reducing protein sample buffer, heated at 95° C. for 5 min, and then subjected to SDS-PAGE followed by coomassie blue staining. Lane M, molecular weight marker; lane 1, VP40-GP VLPs; lane 2, GP virosome preparations.

FIG. 13 illustrates the distribution of Ebola VP40 and GP proteins from different preparations in a sucrose gradient. VLPs or GP virosome preparations (5 ug each) purified from the supernatant of Sf9 cells infected with rBV-VP40 plus rBV-GP as well as rBV-VP40 or rBV-GP alone were loaded on top of a 10-50% sucrose gradient followed by ultracentrifugation. Six 2 ml-fractions (lanes 1 to 6, from the top to the bottom) as well as the pellet (lanes 7) were collected after centrifugation through the sucrose gradient. Proteins in these fractions were concentrated by centrifugation and then analyzed by SDS-PAGE followed by Western blot using a mixture of monoclonal antibodies against Ebola VP40 and GP proteins.

FIG. 14 illustrates bar graphs showing Ebola virosome and VLP stimulation of cytokine secretion by dendritic cells (DCs). Human myeloid DCs were prepared from human PBMC as described in Materials and Methods. DCs were obtained from two healthy donors and incubated with different virosome or VLP preparations in duplicates for each DC preparation. DCs incubated with culture medium only were used as negative controls, and DCs incubated with LPS (10 ng/ml) were used as positive controls. Cell-free supernatants were harvested 24 hr after incubation and assayed for the levels (pg/ml) of TNF-alpha, IL-6, IL10, and IL-12 by ELISA in duplicates. 1, cell culture medium (mock control); 2, 10 ng/ml LPS (positive control); 3, GP virosome preparations (10 ug/ml); 4, VP40 only VLPs (10 ug/ml); 5, Ebola VLPs produced in Sf9 insect cells (10 ug/ml). The results shown represent typical results obtained from two different stimulation experiments and error bars represent standard deviations. Significance of statistical differences in the levels of secreted cytokines stimulated by Ebola VLPs or GP virosome preparations was determined by a t test.

FIG. 15 illustrates bar graphs showing antibody responses against Ebola GP protein induced by immunization with VLPs or GP virosomes. Mice (groups of 6) were immunized intramuscularly with 50 ug of VLP or virosome preparations three times at 4-week intervals. Serum samples were collected at two weeks after each immunization (open bars, after the first immunization; gray bars, after the second immunization; and black bars, after the third immunization) and assayed for Ebola GP specific antibodies by ELISA using purified His-tagged GP1 as coating antigen. A standard curve was generated using purified mouse antibodies for each mouse antibody subtype and used for calculation of the equivalent amount of GP-binding antibodies in serum samples. Group 1, immunized with PBS; group 2, immunized with GP virosome preparations; group 3, immunized with VP40-GP VLPs. Error bars represent standard deviations among individual mouse serum samples in each group. Significance of statistical differences in the levels of antibody responses induced by immunization with VP40-GP VLPs or GP virosome preparations was determined by t test.

FIG. 16 illustrates a graph showing the neutralization of Ebola GP pseudotyped HIV by sera from immunized mice. Serum samples obtained from mice (groups of 6) immunized with PBS, GP virosome preparations, or VP40-GP VLPs at two weeks after the third immunization were heat-inactivated, mixed with 100 pfu of Ebola GP-pseudotyped HIV at 1:40, 1:80, or 1:160 dilutions, and incubated at 37° C. for 1 hr and then added to JC53 cells. At 48 hr post infection, the cells were stained for β-galactosidase expression by addition of X-Gal, and the numbers of blue cells were counted under a light microscope. The percentages of reduction in the number of blue cells in comparison to the control wells were calculated as described in the examples below. Standard deviations for serum samples from each mouse of the group were shown. Significance of statistical differences in neutralizing activity at 1:160 dilution of serum samples from mice immunized with VP40-GP VLPs or GP virosome preparations was determined by a t test.

FIG. 17A illustrates a schematic diagram of an HIV chimeric Env protein construct, M-TM.CT_{MMTV}, in which the signal peptide (SP) was replaced with that of honeybee mellitin, and the transmembrane (TM) and cytoplasmic (CT) domains were replaced with those of mouse mammary tumor virus (MMTV) Env. FIG. 17B illustrates Western blot analysis of the release of the chimeric M-TM.CT_{MMTV} vesicles after sucrose gradient centrifugation. Protein bands were probed with goat anti-gp120 polyclonal antibodies. Lanes 1-11 represent chimeric Env in different fractions of the sucrose gradient from 10 to 60%.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Abbreviations and nomenclature, where employed, are deemed standard in the field and commonly used in professional journals such as those cited herein. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Embodiments of the present disclosure will employ, unless otherwise indicated, techniques of biochemistry, molecular biology, medicine, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Prior to describing the various embodiments, the following definitions are provided and should be used unless otherwise indicated. Other terms may be defined elsewhere in the disclosure, as appropriate.

### Definitions:

The term “organism” or “host” refers to any living entity comprised of at least one cell. A living organism can be as simple as, for example, a single eukaryotic cell or as complex as a mammal, including a human being. As used herein, the term “host” includes humans, mammals (e.g., cats, dogs, horses, chicken, pigs, hogs, cows, and other cattle), and other living species that are in need of treatment. In particular, the term “host” includes humans. Hosts that are “predisposed to” condition(s) can be defined as hosts that do not exhibit overt symptoms of one or more of these conditions but that are genetically, physiologically, or otherwise at risk of developing one or more of these conditions.

The term “treat”, “treating”, and “treatment” are an approach for obtaining beneficial or desired clinical results. Specifically, beneficial or desired clinical results include, but are not limited to, alleviation of symptoms, diminishment of extent of disease, stabilization (e.g., not worsening) of disease, preventing spread of disease, preventing the disease from occurring in an animal that may be predisposed to the disease but does not yet experience or exhibit symptoms of the disease (prophylactic treatment), delaying or slowing of disease progression, amelioration or palliation of the disease state, and remission (partial or total) whether detectable or undetectable. In addition, “treat”, “treating”, and “treatment” can also mean prolonging survival as compared to expected survival if not receiving treatment.

The term “condition” and “conditions” denote a state of health that can be related to infection by a virus. The infections that are discussed herein are to be included as conditions that can be treated by embodiments of the present disclosure.

The term “nucleic acid” or “polynucleotide” is a term that generally refers to a string of at least two base-sugar-phosphate combinations. As used herein, the term includes deoxyribonucleic acid (DNA) and ribonucleic acid (RNA) and generally refers to any polyribonucleotide or polydeoxyribonucleotide, which may be unmodified RNA or DNA or modified RNA or DNA. RNA may be in the form of an tRNA (transfer RNA), snRNA (small nuclear RNA), rRNA (ribosomal RNA), mRNA (messenger RNA), anti-sense RNA, RNAi, siRNA, and ribozymes. Thus, for instance, polynucleotides as used herein refers to, among others, single-and double-stranded DNA, DNA that is a mixture of single-and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The terms “nucleic acid sequence” or “oligonucleotide” also encompasses a nucleic acid or polynucleotide as defined above.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia. For instance, the term polynucleotide includes DNAs or RNAs as described above that contain one or more modified bases. Thus, DNAs or RNAs comprising unusual bases, such as inosine, or modified bases, such as tritylated bases, to name just two examples, are polynucleotides as the term is used herein.

The term also includes PNAs (peptide nucleic acids), phosphorothioates, and other variants of the phosphate backbone of native nucleic acids. Natural nucleic acids have a phosphate backbone; artificial nucleic acids may contain other types of backbones, but contain the same bases. Thus, DNAs or RNAs with backbones modified for stability or for other reasons are “nucleic acids” or “polynucleotides” as that term is intended herein.

“Polypeptide” refers to peptides, proteins, glycoproteins, and the like, of the present disclosure comprising two or more amino acids joined to each other by peptide bonds or modified peptide bonds, (e.g., peptide isosteres). “Polypeptide” refers to both short chains, commonly referred to as peptides, oligopeptides, or oligomers, and to longer chains, generally referred to as proteins.

Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Polypeptides” may contain amino acids other than the 20 gene-encoded amino acids. “Polypeptides” include amino acid sequences modified either by natural processes, such as post-translational processing, or by chemical modification techniques, which are well known in the art. Such modifications are described in basic texts and in more detailed monographs, as well as in a voluminous research literature.

Modifications may occur anywhere in the polypeptides of the present disclosure, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched, and branched cyclic polypeptides may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (Proteins-Structure and Molecular Properties, 2nd Ed., T. E. Creighton, W. H. Freeman and Company, New York, 1993; Wold, F., Post-translational Protein Modifications: Perspectives and Prospects, pgs. 1-12 in Post-translational Covalent Modification of Proteins, B. C. Johnson, Ed., Academic Press, New York, 1983; Seifter, et al., Meth. Enzymol., 182: 626-646, (1990), and Rattan, et al., Ann NY Acad. Sci., 663:48-62, (1992)).

“Variant” refers to polypeptides of the present disclosure that differ from a reference polynucleotide or polypeptide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, and deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity,” as known in the art, is a relationship between two or more polypeptide sequences or two or more polynucleotide sequences, as determined by comparing the sequences. In the art, “identity” also includes the degree of sequence relatedness between polypeptide or polynucleotide sequences, as the case may be, as determined by the match between strings of such sequences. “Identity” and “similarity” can be readily calculated by known methods, including, but not limited to, those described in Computational Molecular Biology, Lesk, A. M., Ed., Oxford University Press, New York, (1988); Biocomputing: Informatics and Genome Projects, Smith, D. W., Ed., Academic Press, New York, (1993); Computer Analysis of Sequence Data, Part I, Griffin, A. M., and Griffin, H. G., Eds., Humana Press, New Jersey, (1994); Sequence Analysis in Molecular Biology, von Heinje, G., Academic Press, 1987; and Sequence Analysis Primer, Gribskov, M. and Devereux, J., Eds., M Stockton Press, New York, (1991); and Carillo, H., and Lipman, D., SIAM J Applied Math., 48, 1073 (1988).

Preferred methods to determine identity are designed to give the largest match between the sequences tested. Methods to determine identity and similarity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 48, 443-453, (1970)) algorithm (e.g., NBLAST, and XBLAST). The default parameters are used to determine the identity for the polynucleotides and polypeptides of the present disclosure.

By way of example, the polypeptide sequences of the present disclosure may be identical to one or more of the reference sequences described above, that is be 100% identical, or it may include up to a certain integer number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “substantially homologous” is used herein to denote polypeptides of the present disclosure having about 50%, about 60%, about 70%, about 80%, about 90%, and preferably about 95% sequence identity to the sequences discussed above. Percent sequence identity is determined by conventional methods as discussed above.

In general, homologous polypeptides of the present disclosure are characterized as having one or more amino acid substitutions, deletions, and/or additions.

In addition, embodiments of the present disclosure include polynucleotides that encode polypeptides having one or more “conservative amino acid substitutions” of the wild type sequence as well as polynucleotides that encode polypeptides that are “functional variants” of the wild type sequence. “Functional variants” includes polypeptides (and polynucleotides encoding such polypeptides) that may have substations, deletions or insertions of more than one amino acid (e.g., substitution of an entire peptide domain or fragment thereof, such as a signal peptide domain, transmembrane domain or cytoplasmic tail domain, of a protein or peptide) but which retains the essential functions of the original, or wild type, protein or peptide.

“Conservative amino acid substitutions” can be based upon the chemical properties of the amino acids. Variants can be obtained that contain one or more amino acid substitutions of the sequences discussed above, in which an alkyl amino acid is substituted for an alkyl amino acid in a polypeptide, an aromatic amino acid is substituted for an aromatic amino acid in a polypeptide, a sulfur-containing amino acid is substituted for a sulfur-containing amino acid in a polypeptide, a hydroxy-containing amino acid is substituted for a hydroxy-containing amino acid in a polypeptide, an acidic amino acid is substituted for an acidic amino acid in a polypeptide, a basic amino acid is substituted for a basic amino acid in a polypeptide, or a dibasic monocarboxylic amino acid is substituted for a dibasic monocarboxylic amino acid in a polypeptide.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamnine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. Other conservative amino acid substitutions include amino acids having characteristics such as a basic pH (arginine, lysine, and histidine), an acidic pH (glutamic acid and aspartic acid), polar (glutamine and asparagine), hydrophobic (leucine, isoleucine, and valine), aromatic (phenylalanine, tryptophan, and tyrosine), and small (glycine, alanine, serine, threonine, and methionine).

Polypeptides having amino acid variants can also comprise non-naturally occurring amino acid residues. Non-naturally occurring amino acids include, without limitation, trans-3-methylproline, 2-4-methanoproline, cis-4-hydroxyproline, trans-4-hydroxyproline, N-methyl-glycine, allo-threonine, methylthreonine, hydroxy-ethylcysteine, hydroxyethylhomocysteine, nitro-glutamine, homoglutamine, pipecolic acid, thiazolidine carboxylic acid, dehydroproline, 3- and 4-methylproline, 3,3-dimethylproline, tert-leucine, norvaline, 2-azaphenyl-alanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations are carried out in a cell-free system comprising an E. coli S30 extract and commercially available enzymes and other reagents. Proteins are purified by chromatography. (Robertson, et al., J. Am. Chem. Soc., 113, 2722, (1991); Ellman, et al., Methods Enzymol., 202, 301, (1991); Chung, et al., Science, 259, 806-9, (1993); and Chung, et al., Proc. Natl. Acad. Sci. USA, 90, 10145-9, (1993)).

In a second method, translation is carried out in Xenopus oocytes by microinjection of mutated mRNA and chemically aminoacylated suppressor tRNAs (Turcatti, et al., J. Biol. Chem., 271, 19991-8, (1996)). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. (Koide, et al., Biochem., 33, 7470-6, (1994)). Naturally occurring amino acid residues can be converted to non-naturally occurring species by in vitro chemical modification. Chemical modification can be combined with site-directed mutagenesis to further expand the range of substitutions (Wynn, et al., Protein Sci., 2, 395-403, (1993)).

“Virosomes” (virosomes) are lipid vesicles having viral envelope proteins expressed on the virosome surface. In addition, adjuvant molecules can be expressed on the virosome. Additional components of virosomes, as known in the art, can be included within or disposed on the virosome. Virosomes do not contain intact viral nucleic acids, and they are non-infectious. Desirably, there are sufficient viral surface envelope glycoproteins and/or adjuvant molecules expressed, at least in part, on the surface of the virosome so that when a virosome preparation is formulated into an immunogenic composition and administered to an animal or human, an immune response (cell-mediated or humoral) is raised.

A “chimeric” virosome, as used herein, can be defined as one or more of the following: a virosome having at least one viral surface envelope glycoprotein incorporated into the virosome and at least one adjuvant molecule, wherein the glycoprotein and the adjuvant molecule are from different sources; a virosome having at least one chimeric viral surface envelope glycoprotein, as defined below, incorporated into the virosome; or a virosome having at least two viral surface envelope glycoproteins incorporated into the virosome where at least one surface envelope glycoprotein is from a different virus than at least one other viral surface envelope glycoprotein.

A “phenotypically mixed” virosome, as used herein, can be defined as a virosome having at least two different surface molecules (e.g., surface envelope glycoproteins and/or adjuvant molecules) incorporated into the virosome. A phenotypically mixed virosome, as used herein, may include virosomes where at least one surface molecule is from a different source than at least one other surface molecule; thus, such a virosome would also be a chimeric virosome, as defined above.

A “truncated” viral surface envelope glycoprotein is one having less than a full length protein (e.g., a portion of the cytoplasmic domain has been removed), but which retains surface antigenic determinants against which an immune response is generated, preferably a protective immune response, and which retains sufficient envelope sequence for proper membrane insertion. The skilled artisan can produce truncated virus envelope proteins using recombinant DNA technology and virus coding sequences, which are readily available to the public.

As used herein “chimeric” viral surface glycoproteins are ones that contain at least a portion of the extracellular domain of a viral surface glycoprotein of one virus and at least a portion of domains and/or a signal peptide sequence of a different transmembrane glycoprotein from a second viral glycoprotein or a cellular protein, such as from a different virus or organism. Such chimeric proteins retain surface antigenic determinants against which an immune response is generated, preferably a protective immune response, and retain sufficient envelope sequence for proper precursor processing and membrane insertion. The skilled artisan can produce chimeric viral surface glycoproteins using recombinant DNA technology and protein coding sequences, techniques known to those of skill in the art and available to the public.

“Expressed”, as used herein, can be defined as being a molecule disposed, or a portion of the molecule disposed, upon the surface of the virosome.

An “expression construct” is an expression vector containing a coding sequence for a recombinant protein.

The term “recombinant” when used with reference to a cell, or nucleic acid, or vector, indicates that the cell, or nucleic acid, or vector, has been modified by the introduction of a heterologous nucleic acid or the alteration of a native nucleic acid, or that the cell is derived from a cell so modified. Thus, for example, recombinant cells express genes that are not found within the native (non-recombinant) form of the cell or express native genes that are otherwise abnormally expressed, under-expressed or not expressed at all. The term “recombinant” generally refers to a non-naturally occurring nucleic acid. Such non-naturally occurring nucleic acids include combinations of DNA molecules of different origin that are joined using molecular biology technologies, or natural nucleic acids that have been modified, for example that have deletions, substitutions, inversions, insertions, etc. Recombinant also refers to the polypeptide encoded by the recombinant nucleic acid. Non-naturally occurring nucleic acids or polypeptides include nucleic acids and polypeptides modified by man.

The term “heterologous” indicates derived from a separate genetic source, a separate organism, or a separate species. Thus, a heterologous antigen is an antigen from a first genetic source expressed by a second genetic source. The second genetic source is typically a vector.

The term “operably linked” refers to the arrangement of various nucleotide sequences relative to each other such that the elements are functionally connected to and are able to interact with each other. Such elements may include, without limitation, one or more promoters, enhancers, polyadenylation sequences, and transgenes. The nucleotide sequence elements, when properly oriented, or operably linked, act together to modulate the activity of one another, and ultimately may affect the level of expression of the transgene. For example, control sequences or promoters operably linked to a coding sequence are capable of effecting the expression of the coding sequence, and an organelle localization sequence operably linked to protein will direct the linked protein to be localized at the specific organelle. The position of each element relative to other elements may be expressed in terms of the 5′ terminus and the 3′ terminus of each element, and the distance between any particular elements may be referenced by the number of intervening nucleotides, or base pairs, between the elements.

A “vector” is a genetic unit (or replicon) to which or into which other DNA segments can be incorporated to effect replication, and optionally, expression of the attached segment. Examples include, but are not limited to, plasmids, cosmids, viruses, chromosomes and minichromosomes. Exemplary expression vectors include, but are not limited to, baculovirus vectors, modified vaccinia Ankara (MVA) vectors, plasmid DNA vectors, recombinant poxvirus vectors, bacterial vectors, recombinant baculovirus expression systems (BEVS), recombinant rhabdovirus vectors, recombinant alphavirus vectors, recombinant flavivirus vectors, recombinant paramyxovirus vectors, recombinant adenovirus expression systems, recombinant herpesvirus vectors, recombinant DNA expression vectors, and combinations thereof.

A “coding sequence” is a nucleotide sequence that is transcribed into mRNA and translated into a protein, in vivo or in vitro.

“Regulatory sequences” are nucleotide sequences, which control transcription and/or translation of the coding sequences that they flank.

“Processing sites” are described in terms of nucleotide or amino acid sequences (in context of a coding sequence or a polypeptide). A processing site in a polypeptide or nascent peptide is where proteolytic cleavage occurs, where glycosylation is incorporated or where lipid groups (such as myristoylation) occurs. Proteolytic processing sites are where proteases act.

The term “antibody” is used to refer both to a homogenous molecular entity, or a mixture such as a serum product made up of a plurality of different molecular entities. Monoclonal or polyclonal antibodies, which specifically react with the virosomes of the present disclosure, may be made by methods known in the art. (e.g., Harlow and Lane (1988) Antibodies: A Laboratory Manual, Cold Spring Harbor Laboratories; Goding (1986) Monoclonal Antibodies: Principles and Practice, 2d ed., Academic Press, New York; and Ausubel et al. (1987)). Also, recombinant immunoglobulin may be produced by methods known in the art, including but not limited to, the methods described in U.S. Pat. No. 4,816,567, which is hereby incorporated by reference herein.

“Immunogenic compositions” are those which result in specific antibody production or in cellular immunity when injected into a host. Such immunogenic compositions or vaccines are useful, for example, in immunizing hosts against infection and/or damage caused by viruses, including, but not limited to, HIV, human T-cell leukemia virus (HTLV) type I, SIV, FIV, SARS, RVFV, Filovirus, Flavivirus, arenavirus, bunyavirus, paramyxovirus, influenza virus, cytomegalovirus, herpesvirus, alphavirus, and flavivirus.

By “immunogenic amount” is meant an amount capable of eliciting the production of antibodies directed against the virus, in the host to which the vaccine has been administered.

“Pharmaceutically acceptable salts” include, but are not limited to, the acid addition salts of compounds of the present disclosure (formed with free amino groups of the peptide) which are formed with inorganic acids (e.g., hydrochloric acid or phosphoric acids) and organic acids (e.g., acetic, oxalic, tartaric, or maleic acid). Salts formed with the free carboxyl groups may also be derived from inorganic bases (e.g., sodium, potassium, ammonium, calcium, or ferric hydroxides), and organic bases (e.g., isopropylamine, trimethylamine, 2-ethylamino-ethanol, histidine, and procaine).

As used herein, a “pharmaceutically acceptable carrier” refers to a carrier or diluent that does not cause significant irritation to an organism and does not abrogate the biological activity and properties of the administered compound.

An “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

### Discussion:

Embodiments of the present disclosure provide for virosomes, methods of using the virosomes, and methods of making virosomes that can be used in immunogenic compositions to treat conditions in a host, and the immunogenic compositions that include virosomes. The virosomes can be used to enhance immune responses (e.g., antibody production, cytotoxic T cell activity, and cytokine activity). In particular, virosomes can act as a prophylactic as a vaccine to prevent viral infections such as those caused by, for example, the human immunodeficiency virus (HIV), the Corona virus, the Influenza virus, the Paramyxovirus, the Herpesvirus, the Ebola virus, the Rift Valley Fever virus (RVFV), the Hantaan Virus, the Lassa fever virus, and the Flavivirus, among others.

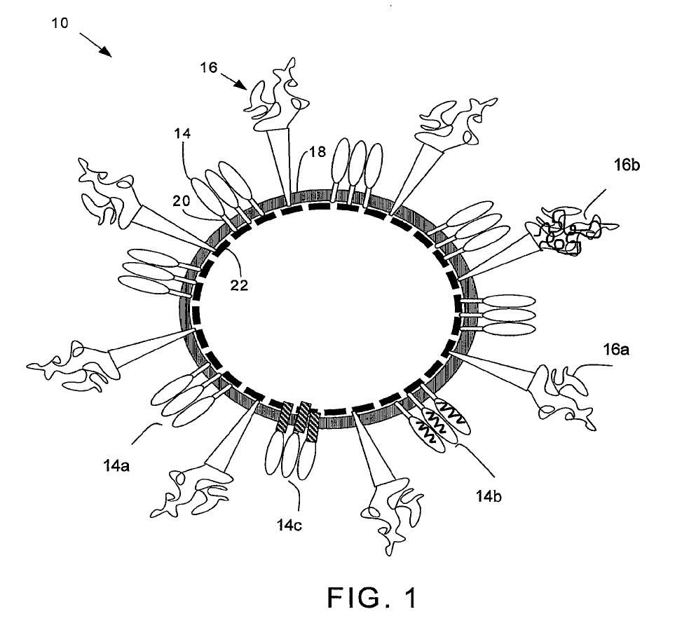

As illustrated in FIG. 1, the virosome 10 includes at least one viral surface envelope glycoprotein 14 (e.g., a RVFV glycoprotein, an Ebola glycoprotein, a chimeric viral surface envelope glycoprotien, etc.) and, optionally, one or more adjuvant molecules 16. In exemplary embodiments, the virosome includes at least two different viral surface envelope glycoproteins 14 (e.g., type 1 (14a) and type 2 (14b), and so on) and, optionally, one or more adjuvant molecules 16. In other exemplary embodiments, the virosome includes at least one chimeric viral surface envelope glycorotein 14c and, optionally, one or more adjuvant molecules 16. In yet other exemplary embodiments, the virosome includes at least one viral surface envelope glycoprotien 14 and at least one adjuvant molecule 16. The adjuvant molecules 16 can include more than one type of adjuvant molecule (e.g. 16a and 16b, and so on).

Furthermore, the virosome can also include a lipid membrane 18 and a viral glycoprotein transmembrane unit 20. In particular, chimeric virosomes are virosomes having at least one chimeric viral surface envelope glycoprotein 14c or having at least two viral surface envelope glycoproteins 14, where at least one viral surface envelope glycoprotein 14a is from a different virus than at least one other viral surface envelope glycoprotein 14b. A chimeric viral surface envelope glycoprotein (e.g., 14c) includes at least a portion of a viral surface envelope glycoprotein from one virus and at least a portion of a viral surface envelope glycotprotein from a second viral glycoprotein or a cellular protein, as will be described in greater detail below.

Furthermore, phenotypically mixed virosomes include virosomes having at least one adjuvant molecule 16 incorporated into the virosome, where at least one of the adjuvant molecule(s) 16 have an origin different from that of at least one viral surface envelope glycoprotein 14. Phenotypically mixed virosomes also include virosomes where there are more than one type of adjuvant molecule 16 (16a and 16b) and where one or both of 16a and 16b are from a different source from each other.

The viral surface envelope glycoprotein 14, or at least at portion of the viral surface envelope glycoprotein 14, is disposed (e.g., expressed) on the surface of the virosome. The viral surface envelope glycoprotein 14 is disposed on the surface of the virosome so that it can interact with target molecules or cells (e.g., the interaction between the HIV surface envelope glycoprotein and the B cell receptor to activate HIV envelope glycoprotein specific antibody producing B cells) to produce immunogenic responses (e.g., antibody production).

The viral surface envelope glycoproteins 14 can include, but are not limited to, a retrovirus glycoprotein (e.g., a human immunodeficiency virus (HIV) envelope glycoprotein (e.g., HIVSF162 envelope glycoprotein (SEQ ID NO: 1, GenBank serial number M65024)), a simian immunodeficiency virus (SIV) envelope glycoprotein (e.g., SIVmac239 envelope glycoprotein (GenBank serial number M33262)), a simian-human immunodeficiency virus (SHIV) envelope glycoprotein (e.g., SHIV-89.6p envelope glycoprotein (GenBank serial number U89134)), a feline immunodeficiency virus (FIV) envelope glycoprotein (e.g., feline immunodeficiency virus envelope glycoprotein (GenBank serial number L00607)), a feline leukemia virus envelope glycoprotein (e.g., feline leukemia virus envelope glycoprotein (GenBank serial number M12500)), a bovine immunodeficiency virus envelope glycoprotein (e.g., bovine immunodeficiency virus envelope glycoprotein (GenBank serial number NC001413)), a bovine leukemia virus envelope glycoprotein (GenBank serial number AF399703), a equine infectious anemia virus envelope glycoprotein (e.g., equine infectious anemia virus envelope glycoprotein (GenBank serial number NC001450)), a human T-cell leukemia virus envelope glycoprotein (e.g., human T-cell leukemia virus envelope glycoprotein (GenBank serial number AF0033817)), and a mouse mammary tumor virus envelope glycoprotein (MMTV)); a bunyavirus glycoprotein (e.g., a Rift Valley Fever virus (RVFV) glycoprotein, (e.g., RVFV envelope glycoprotein (SEQ ID NO: 2, GenBank serial number M11157))); an arenavirus glycoprotein (e.g., a Lassa fever virus glycoprotein (GenBank serial number AF333969))); a filovirus glycoprotein (e.g., an Ebola virus glycoprotein (GenBank serial number NC002549)); a corona virus glycoprotein (GenBank serial number SARS coronavirus spike protein AAP13567); an influenza virus glycoprotein (GenBank serial number V01085)); a paramyxovirus glycoprotein (GenBank serial number NC002728 for Nipah virus F and G proteins); a rhabdovirus glycoprotein (GenBank serial number NP049548)) (e.g., a Vesicular Stomatitis Virus (VSV) glycoprotein); an alphavirus glycoprotein (GenBank serial number AAA48370 for Venezuelan equine encephalomyelitis (VEE)); a flavivirus glycoprotein (GenBank serial number NC001563 for West Nile virus) (e.g., a Hepatitis C Virus glycoprotein); a Herpes Virus glycoprotein (e.g., a cytomegalovirus glycoprotein, and a herpes simplex virus glycoprotein); a respiratory syncytial virus glycoprotein; and combinations thereof.

In general, the viral surface envelope glycoprotein 14 sequence and the corresponding polynucleotide sequence can be found in GenBank, and the access numbers can be obtained online at NCBI. In addition, the sequences identified for the viral surface envelope glycoproteins 14 above are only illustrative examples of representative viral surface envelope glycoproteins 14. Further, variants that are substantially homologous to the above referenced viral surface envelope glycoproteins 14 and viral surface envelope glycoproteins 14 having conservative substitutions of the above referenced viral surface envelope glycoproteins 14 can also be incorporated into virosomes 10 of the present disclosure to enhance the immunogenic characteristics of virosomes.

In one embodiment, the viral surface envelope glycoprotien (e.g., an HIV envelope glycoprotein) can be modified and/or truncated to improve the immunogenic properties of the virosome. Also, the virosome can be conformationally changed by hydrostatic pressure-induced techniques.

In an exemplary embodiment, the HIV envelope glycoprotein can be modified to expose neutralizing epitopes in the HIV envelope glycoprotein by removing obstructing structural features such as, but not limited to, glycosylation sites, the V1 loop, the V2 loop, and the V3 loop. By eliminating these obstructing features, the immunogenic properties of the virosome that includes such modified glycoproteins can be enhanced.

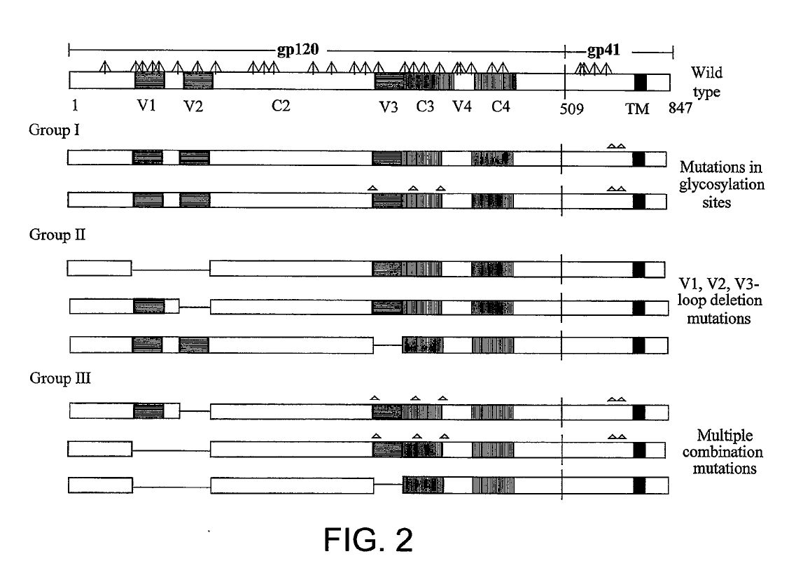

FIG. 2 illustrates some representative structural changes that can be made to the HIV 89.6 envelope glycoprotein (GenBank serial number AAA81043, SEQ ID NO: 3). The arrows in FIG. 2 indicate the N-glycosylation motifs in the HIV 89.9 viral surface envelope glycoprotein as well as the V1 (amino acids 128-164 of SEQ ID NO: 3) loop, V2 (amino acids 164-194 of SEQ ID NO: 3) loop, and V3 (amino acids 298-329 of SEQ ID NO: 3) loop domains. Deletions of the loops is shown by removing the corresponding sequence in the HIV 89.6 envelope glycoprotein shown in FIG. 2. When the loop is deleted, a 3 amino acid linker (GAG) is inserted into the loops former position in the sequence. The mutated glycosylation motifs are denoted by triangles. Three glycosylation motif mutations of Asn to Gln in the V3/C3 (amino acids 298-402 of SEQ ID NO: 3) domains are performed on amino acids 301, 341, and 362. In the gp41 domain (amino acids 509-853 of SEQ ID NO: 3), the glycosylation motif mutations are performed on Asn in amino acid position 623 and 635. TM denotes the transmembrane domain (amino acids 681-7033 of SEQ ID NO: 3). In FIG. 2, Group I illustrates the glycosylation motif mutations in gp41, the V1 loop domains, the V2 loop domain, and the V3-C3 loop domain. Group II illustrates variable loop (V1, V2, and/or V3) deletion mutations, while Group III illustrates representative multiple combination mutations.

For example, mutations of glycosylation sites in gp41 can be performed to enhance the immunogenic properties of a virosome incorporating the HIV envelope glycoprotein. Although most of gp41 appears to be completely occluded in the HIV-1 envelope spike, recent studies indicate that regions of gp41 close to the transmembrane domain are accessible to neutralizing antibodies (Abs). Several mAbs (2F5, Z13, 4E10), which neutralize a broad range of primary HIV-1 isolates, are known to bind to the extracellular domain of gp41 . However, attempts to elicit antibodies having these properties by immunization with linear peptide epitopes or with other carrier proteins containing peptide epitopes have not been successful (Coeffier et al., Vaccine, 19, 684-693, (2000); Eckhart, L., et al., J. Gen. Viro., 77 (Pt. 9), 2001-2008, (1996); and Liang, X., et al., Vaccine, 17, 2862-2872, (1999)). These studies indicate that the introduction of neutralizing antibodies against gp41 epitopes may be dependent on the native form of trimeric gp120-gp41 and that these epitopes may not be immunodominant, possibly due to the presence of N-glycans. As shown in FIG. 2, the gp41 domain in the HIV-1 envelope glycoprotein contains four conserved glycosylation motifs (#22 (amino acid 608 of SEQ ID NO: 3), #23 (amino acid 613 of SEQ ID NO: 3), #24 (amino acid 622 of SEQ ID NO: 3), and #25 (amino acid 634 of SEQ ID NO: 3). Viruses with single or double mutations in these glycosylation sites have been replicated in both human and monkey T cell lines (Johnson, W. E., et al., J. Virol., 75, 11426-11436, (2001), which is hereby incorporated by reference herein). Removing the glycosylation motifs #24 (amino acid 622 of SEQ ID NO: 3) and #25 (amino acid 634 of SEQ ID NO: 3) near the neutralizing epitopes may increase the exposure of these epitopes and thus, enhance the induction of neutralizing antibodies against the gp41 domain (numbering glycosylation motifs from the N-terminus of the HIV 89.6 envelope glycoprotein).

It should also be noted that, upon binding of the HIV envelope glycoprotein to its receptor molecules or the shedding of its surface subunit (gp120), its transmembrane subunit (gp41 ) converts into a six-helix bundle configuration, which is highly immunogenic but only presents non-neutralizing epitopes (Kim, P. S., Annual Reviews of Biochemistry, 70, 777-810, (2001)). The use of a peptide containing the amino acid sequence corresponding to a segment of the gp41 (amino acids 629 to 664 of SEQ ID NO: 1) can effectively block the transition into the undesirable six-helix bundle configuration. Therefore, treatment of virosomes with such a peptide can help to preserve the HIV envelope glycoprotein on the surface of the virosome to retain its native configuration for more efficient exposure of neutralizing epitopes and thus the induction of neutralizing antibodies. Such structural features are common to the envelope glycoproteins of many viral families, including, but not limited to, the envelope glycoproteins of retroviruses, influenza virus, and parainfluenza virus. Thus, such a virosome treatment approach can be applied to a variety of virosome vaccines.

In another example, the V1 loop, the V2 loop, and the V3 loop can be deleted to enhance the immunogenic properties of virosomes. Deletion of individual V1 or V2 loops does not reduce the potential of the virus to replicate in PBMCs or alter the co-receptor binding of the viral surface envelope glycoprotein (Stamatatos, L., et al., Aids Res. Hum. Retroviruses, 14, 1129-1139, (1998)). HIV-1 mutants lacking the V1 and V2 loops in gp120 exhibited increased sensitivity to neutralization by antibodies directed against V3 and a CD4-induced epitope on gp120, and by sera collected from patients infected with clades B, C, D, and F HIV-1 primary isolates (Cao, J., et al., J. Virol., 71, 9808-9812, (1997) and Stamatatos, L., and C. Cheng-Mayer, J. Virol., 72, 7840-7845, (1998)). These studies suggest that the V2 loop or V2 together with the V1 loop shields some important neutralization epitopes with an overall structure that appears to be conserved among different HIV-1 primary isolates. Thus, deleting the V1-V2 loop or V2 loop may expose hidden neutralizing epitopes. Such mutant glycoproteins can be incorporated into virosome vaccines.

The V3 loop of the HIV envelope glycoprotein is highly variable and also constitutes a dominant epitope for the antibody response. Although neutralizing antibodies against this region are frequently detected, they are often strain-specific (Sattentau, Q. J., et al., Virol., 206(1), 713-7, (1995) and D'Souaza, M. P., et al., Aids, 9, 867-874, (1995)). Furthermore, deletion of the V3 loop has also been shown to increase the exposure of epitopes induced by sCD4 binding (Sanders, R. W., et al., J. Virol., 74, 5091-5100, (2000)). Lu et al. (Aids Res. Hum. Retroviruses, 14, 151-155, (1998)) compared antibody induction by gene-gun immunization of rabbits with DNA vectors expressing HIV-1 IIIB Gp160, Gp140, Gp120 and their corresponding V1/V2/V3 triple loop deletion mutants. These results showed that deletion of variable loops induced higher ELISA antibody responses but not neutralizing antibody responses. Such mutant glycoproteins can also be incorporated into virosome vaccines.

In another study, Garrity et al. (J. Immunol., 159, 279-289, (1997)) showed that immunization of guinea pigs using recombinant vaccinia virus followed by protein boosting with mutant viral surface envelope glycoprotein 120, in which glycosylation sites were introduced to mask the immunodominant domain in the V3 loop, was more effective in inducing cross-reactive neutralizing antibodies against a divergent strain of the same subtype. Thus, eliminating the immunodominant epitopes in the V3 loop may enhance induction of cross-reactive antibodies. Furthermore, Kiszka et al. (J. Virol., 76, 4222-4232, (2002)) reported that immunization of mice using DNA vaccines encoding HIV envelope glycoproteins with V3 loop deletions induced broader cellular immune responses to subdominant epitopes and was more effective in conferring protection against challenge with recombinant vaccinia virus expressing heterologous HIV envelope glycoproteins, indicating that deletion of the V3 loop may also be advantageous in inducing broader cellular immune response. Such a deletion mutant glycoprotein can be incorporated into virosome vaccines.

In another embodiment, the virosome includes a RVFV envelope glycoprotein, which can include, but is not limited to, a RVFV GC envelope glycoprotein (SEQ ID NO: 4) and a RVFV GN envelope glycoprotein (SEQ ID NO: 5). The RVFV GC and GN envelope glycoproteins can be modified to enhance the immunogenic properties of the virosome 10. For example, the RVFV GC and GN envelope glycoproteins can be modified by truncating the cytoplasmic domain for the RVFV GC (amino acids 492-507 of SEQ ID NO: 4) and GN envelope glycoproteins (amino acids 458-527 of SEQ ID NO: 5).

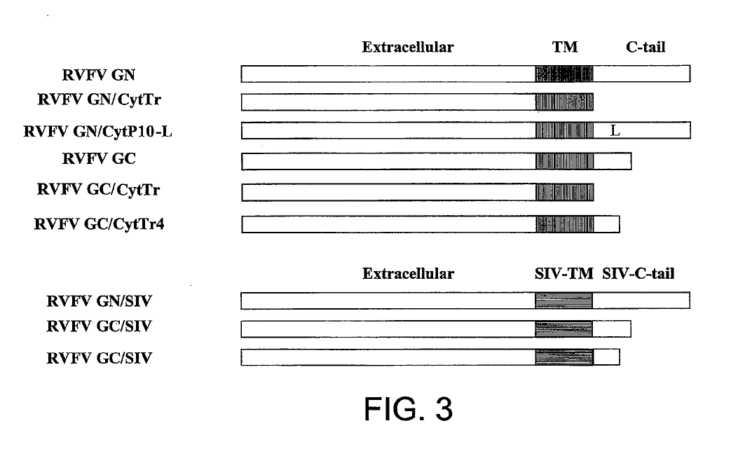

FIG. 3 illustrates some representative structural changes that can be made to the RVFV GN and GC envelope glycoproteins. For example, the RVFV GC and GN envelope glycoproteins can be modified by truncating the cytoplasmic domain for the RVFV GC (amino acids 492-507 of SEQ ID NO: 4) and GN envelope glycoproteins (amino acids 458-527 of SEQ ID NO: 5). In addition, the RVFV GN envelope glycoprotein can mutated by replacing the proline residue (amino acid 537 of SEQ ID NO: 5) from the cytoplasmic domain (conserved between RVFV and PTV GN envelope glycoprotein) with a leucine residue. Such modifications could increase levels of surface expression of the RVFV envelope glycoproteins and therefore increase their incorporation into virosomes. Thus, the effectiveness of the virosomes to elicit immune response against RVFV envelope glycoproteins may be enhanced since the virosomes may contain more RVFV envelope glycoproteins per unit amount.

Furthermore, the RVFV GC and GN envelope glycoproteins can be modified by replacing the transmembrane domain and/or the cytoplasmic tails of the RVFV GC and GN envelope glycoproteins with the transmembrane domain and the cytoplasmic tail of the SIV envelope glycoprotein. Studies on retrovirus assembly have shown that efficient incorporation of viral surface envelope glycoproteins may involve specific interaction between viral Gag proteins and the cytoplasmic domain of the viral surface envelope glycoprotein (Cosson, P., et al., EMBO J., 15, 5783-5788, (1996); Vincent, M. J., et al., J. Virol., 73, 8138-44, (1999); and Wyma, D. J., et al., J. Virol., 74, 9381-7, (2000)). Therefore, replacing the transmembrane domain and cytoplasmic tails of RVFV GN and GC envelope glycoproteins with those of the HIV or SIV envelope glycoprotein (SEQ ID NO: 6) or cytoplasmic tails (SEQ ID NO: 8) may produce chimeric proteins with increased cell surface expression and more efficient incorporation into SIV virosomes.

In previous studies, SIV envelope glycoproteins containing such a truncated cytoplasmic domain yielded high levels of cell surface expression and more efficient incorporation into SIV Virus Like Particles (VLPs) in comparison to the SIV envelope glycoprotein with a full length cytoplasmic domain of over 150 amino acids (Vzorov, A. and Compans, R. W., J. Virol., 74, 8219-25, (2000)). Furthermore, the cytoplasmic domain of the SIV envelope glycoprotein contains a Tyr-based endocytosis signal, which has been shown to induce rapid endocytosis of the SIV envelope glycoprotein and lead to reduced surface expression (Labranche, C. C., et al., J. Virol., 69, 5217-5227, (1995)). Thus, attaching a truncated SIV envelope glycoprotein cytoplasmic domain to the RVFV envelope glycoproteins that are expressed on the cell surface may produce enhanced incorporation into virosomes.

In addition, a Tyr residue can be replaced by other amino acids, including Cys, in the attached SIV cytoplasmic domain sequence (e.g., amino acid 16 of SEQ ID NO: 8) to further augment surface expression of designed chimeric proteins. This design for chimeric proteins can be applied to both RVFV GN and GC envelope glycoproteins. Such modifications may increase levels of surface expression of the RVFV envelope glycoproteins and therefore increase their incorporation into virosomes. Thus, the effectiveness of the virosomes to elicit immune response against RVFV envelope glycoproteins may be enhanced, since the virosomes contain more RVFV envelope glycoproteins per unit amount.

In some embodiments, an adjuvant molecule 16, or at least a portion of an adjuvant molecule 16, is disposed (e.g., expressed) on the surface of the virosome 10. The adjuvant molecule 16 can interact with other molecules or cells (e.g., mucosal surfaces having sialic acid residues disposed thereon and antigen-presenting cells such as dendritic cells and follicular dendritic cells).

The adjuvant molecule 16 can include, but is not limited to, an influenza hemagglutinin (HA) molecule (GenBank access number J02090), a parainfluenza hemagglutinin-neuraminidase (HN) molecule (GenBank access number z26523 for human parainfluenza virus type 3 HN sequence information), a Venezuelan equine encephalitis (VEE) adjuvant molecule (GenBank access number nc001449), a fms-like tyrosine kinase ligand (Flt3) adjuvant molecule (GenBank access number NM013520), a C3d adjuvant molecule (GenBank access number nm009778 for mouse C3 sequence and access number nm000064 for human C3 sequence), a mannose receptor adjuvant molecule, a CD40 ligand adjuvant molecule (GenBank access number m83312 for mouse CD40), and combinations thereof. The adjuvant molecule 16 can also include membrane anchored forms of a mammalian toll-like receptor (TLR) ligand molecule, a MIP-1α molecule, a RANTES MIP-1β molecule, a GM-CSF molecule, a Flt3 ligand molecule, a CD40 ligand molecule, an IL-2 molecule, an IL-10 molecule, an IL-12 molecule, an IL-15 molecule, an IL-18 molecule, and an IL-21 molecule, and combinations thereof. Examples of membrane-anchored forms of mammalian TLR ligand molecules include, but are not limited to, ligands listed in Akira, S. and Takeda, K. Toll-Like Receptor Signalling. Nature Reviews/Immunology, 4: 499-511 (2004), which is incorporated by reference herein. In particular, exemplary TLR ligand molecules include glycoproteins from Prevotella intermedia, Respiratory syncytial virus protein F, fibronectin A domain, fibrinogen, a baceterial flagellin, a measles virus HA protein, and Pam2Cys lipoprotein/lipopeptide (MALP-2). In some particular embodiments the adjuvant molecule includes a membrane-anchored bacterial flagellin.

In general, the adjuvant molecule 16 sequence and the corresponding polynucleotide sequence can be found in GenBank, and the access numbers can be obtained online at the NCBI. In addition, the sequences identified for the adjuvant molecules 16 above are only illustrative examples of representative adjuvant molecules 16. Further, variants that are substantially homologous to the above referenced adjuvant molecules 16 and adjuvant molecules 16 having conservative substitutions of the above referenced adjuvant molecules 16 can also be incorporated into virosomes 10 of the present disclosure to enhance the immunogenic characteristics of virosomes.

Mucosal immunity is important for prevention of infection by aerosolized virus because mucosal antibodies can neutralize the virus and/or block virus attachment of the virus to the mucosal cells with secreted antibodies. However, little success has been documented for eliciting strong mucosal immune responses by non-replicating vaccines against viruses other than influenza, which is attributed, at least in part, to the difficulty of targeting the antigens to mucosal sites. In contrast, inactivated influenza virus has been shown to induce strong mucosal immune responses when administered mucosally, which may be the result of the strong binding affinity of the HA adjuvant molecule for sialic acid residues that are abundant at mucosal surfaces. Therefore, the affinity of HA adjuvant molecule for sialic acids may be utilized for targeting virosomes to mucosal surfaces. Incorporating HA adjuvant molecules into virosomes may enhance the immunogenic properties of virosomes.

Mucosal immune responses against HIV play an important role in prevention of HIV infection and transmission, as the mucosal surface is the major site for initial HIV infection. Being the first line of defense, mucosal immunity is critical for prevention of infection by neutralizing virus and/or blocking virus attachment with secreted antibodies. However, little success has been documented for eliciting strong mucosal immune responses against HIV, which is attributed at least in part to the difficulty of targeting the antigens to mucosal sites. In contrast, inactivated influenza virus has been shown to induce strong mucosal immune responses when administered mucosally, a likely result of the strong binding affinity of its HA adjuvant molecule for sialic acid residues that are abundant on mucosal surfaces. Therefore, chimeric virosomes incorporating the HA adjuvant molecule may be suited to target mucosal surfaces since the HA adjuvant molecule has an affinity for sialic acid. Such virosomes may thus be used for the mucosal immunization against HIV.

The possibility of pre-existing immunity to the influenza HA protein is a factor that should be considered in the evaluation of HA-virosome vaccines. However, it is uncertain that the existence of such preexisting immunity would negatively impact the immune responses to the virosomes (in marked contrast to immunization using replicating vectors). It is possible that preexisting antibodies would lead to production of immune complexes that would enhance targeting of virosomes to follicular dendritic cells and thus result in stimulation of B cell responses.

Nevertheless, alternative approaches can be applied for mucosal targeting of virosomes. One possibility is to utilize influenza HA adjuvant molecules from other influenza virus species to which there is no preexisting immunity in the human population. About 15 such non-cross reactive serotypes of HA adjuvant, which are antigenically non-overlapping based on tests with polyclonal immune sera, molecules have been identified. The 15 non-cross reactive serotypes of HA viruses and their replication are described in the following publications: Lamb, R. A. and Krug, R. M., Orthomyxoviridae, (1996) and Fields, B. N., et. al., Editors, Field's Virology, Lippincott-Raven Publishers, Philadelphia, Pa., 1353-1395, (1996). Thus, virosomes can be produced containing one or more of these alternative HA adjuvant molecule subtypes, therefore avoiding a possible effect of preexisting anti-HA immunity on induction of immune responses against virosome antigens. It should be noted that some influenza HA adjuvant molecules from other species may bind preferentially to sialic acid linkages not found on human cells. This property, however, can be modified by mutation of specific HA amino acids (Vines, A., et al., J. Virol., 72, 7626-7631, (1998)).

An alternative approach to using HA adjuvant molecules is the production of virosomes containing parainfluenza virus HN adjuvant molecules. Like HA adjuvant molecules, the HN adjuvant molecules attach specifically to the sialic acid residues at mucosal surfaces. Therefore, chimeric virosomes containing HN adjuvant molecules should have similar mucosal targeting properties as the HA adjuvant molecules. However, immune responses to the proteins of human parainfluenza viruses are of relatively short duration, and reinfections with the same viral serotypes are known to occur (Glezen, W. P., et al., J. Infect. Dis., 150, 851-857, (1984)). Thus, as compared with HA, it is less likely that preexisting immunity to the HN adjuvant molecules of a parainfluenza virus would affect mucosal delivery of a virosome vaccine.

HN-virosomes may be easier to produce in modified vaccinia Ankara expression systems rather than HA-virosomes. This is because the release of HA chimeric virosomes from mammalian cells would require addition of exogenous neuraminidase (Bosch, V., et al., J. Gen. Virol., 82, 2485-2494, (2001)) since sialic acid would be added to the envelope glycoproteins as a terminal sugar and lead to aggregation of virosomes at the cell surface (which does not occur in the insect cell-produced virosomes). In contrast, HN carries its own neuraminidase. Studies of viral pseudotypes have shown that the glycoproteins of parainfluenza viruses including the HN adjuvant molecule can be assembled into virions of retroviruses, indicating that this type II membrane protein can be incorporated into virosomes (Spiegel, M. et al., J. Virol., 72, 5296-5302, (1998)). Thus, incorporating HN adjuvant molecules into virosomes may enhance the immunogenic properties of virosomes.

Antigen presenting cells can be targeted by virosomes by including, but not limited to, one or more of the following adjuvant molecules on the surface of the virosome: the VEE adjuvant molecule, the Flt3 ligand molecule, the mannose adjuvant molecule, the CD40 adjuvant molecule, and the Cd3 ligand molecule. In particular, the VEE adjuvant molecule, the Flt3 adjuvant molecule, the mannose receptor adjuvant molecule, and the CD40 adjuvant molecule can be used to target dendritic cells, while the Cd3 ligand molecule can be used to target follicular dendritic cells.

Dendritic cells (DCs) are very efficient antigen presenting cells involved in priming native CD4 and CD8 T cells, thus inducing primary immune responses and permitting establishment of immunological memory (Inaba, K., et al., J. Exp. Med., 166:182-194, (1987) and Inaba, K., et al., J. Exp. Med., 172:631-640, (1990)). Antigens taken up by DCs are expressed at the cell surface in the form of peptides associated with MHC class II, which stimulates CD4 Th cells. For induction of CD8 T cells, MHC class I associated peptides are derived from endogenously synthesized proteins as well as from some exogenous antigens (e.g., infectious agents, dying cells, proteins associated with inert particles, and immune complexes) by DC endocytosis (Heath, W. R. and F. R. Carbone, Curr. Opin. Immunol., 11:314-318, (1999); Reimann, J. and R. Schirmbeck, Immunol. Rev., 172; 131-152, (1999); Regnault, A., et al., J. Exp. Med., 189:371-380, (1999); and Machy, P., et al., Eur. J. Immunol., 30:848-857, (2000)). DCs harboring immune complexes also stimulate naive B cells (Wykes, M., J. Immunol., 161, 1313-1319, (1998) and Dubois, B., et al., Biol., 70, 633-641, (2001)). The highly developed Ag-presenting capacity of DCs has led to their study as cellular vaccine adjuvants for the immunotherapy of cancer (Schuler, G. and R. M. Steinman, J. Exp. Med., 1986: 1183-1187, (1997) and Baggers, J., et al., J. Clin. Oncol., 18:3879-3882, (2000)). HIV and SIV virions interact with DCs via DC-SIGN and/or CD4 receptors; however, this interaction appears to preferentially result in infection of the DCs as well as transmission to other target cells rather than potentiation of an immune response (Geijtenbeek, T. B., et al., Cell, 100: 587-597, (2000) and Geijtenbeek, T. B., et al., Immunol. Lett. 79:101-107, (2001)). On the other hand, inert particulate antigens like virosomes are very attractive target for antigen presenting cells, particularly DCs (Bachmann, M. F., et al., Eur. J. Immunol., 26:2595-2600, (1996); Ruedl, C., et al., Eur. J. Immunol., 32:818-825, (2002) and Da Silva, D. M., et al., Int. Immunol., 13:633-641, (2001)). Therefore, the interaction of virosomes with DCs may result in potentiating DCs to initiate T cell activation.

The possible advantage of targeting vaccine antigens to DCs is indicated by the extremely small number of DCs in peripheral tissues and in blood, where DCs represent less than 1% of total cell number. Flt3 ligand (FL) adjuvant molecule (GenBank access number NM013520) is a hematopoietic growth factor that has the unique ability to expand the number of both CD8α- and CD8α+ DC subsets (Lyman, S. D., et al., Cell, 75:1157-1167, (1993); Maraskovsky, E, et al., J. Exp. Med., 184:1953-1962, (1996); Maraskovsky, E, et al., Blood, 96:878-884, (2000) and Pulendran, B., et al., J. Immunol., 159:222-2231, (1997)). Such expansion of DCs in mice resulted in dramatic increases in Ag-specific B and T cell responses (Pulendran, B., et al., J. Exp. Med., 188, 2075-2082, (1998)), enhanced T-cell mediated immune responses (Pisarev et al., Int J Immunopharmacol, 11, 865-76, (2000)), and protective immunity to Listeria monocytogenes (Gregory, S. H., et al., Cytokine, 13:202-208, (2001)). It is suggested that FL treatment increases the capacity of DCs as antigen presenting cells by up-regulating MHC and costimulatory molecules (CD40, CD86), and by inducing production of cytokines (IFN-y, IL-2, IL-12 or IL-4) (Pulendran, B., et al., J. Exp. Med., 188, 2075-2082, (1998) and Pulendran, B., et al., Proc. Natl. Acad. Sci., U.S.A. 96, 1036-1041, (1999)). Therefore, incorporation of FL adjuvant molecules into virosomes may enhance the immunogenic properties of the virosomes.

In an exemplary embodiment, the virosome can be produced to include the FL adjuvant molecule by PCR-amplifying and cloning the whole FL gene including the signal sequence and transmembrane (TM) domain into rBV transfer vector pc/pS1. To construct a rBV expressing FL, Sf9 insect cells can be co-transfected with Baculogold DNA (available from PharMingen, Inc.) and the pc/pS1 transfer vector containing the FL gene.

The incorporation of the FL adjuvant molecule into virosomes can be enhanced by modifying the FL adjuvant molecule. In particular, the extracellular coding domain of the FL gene (from the end of signal peptide to the start of the TM segment) (SEQ ID NO: 7) can be fused to the N-terminus of the SIV Env glycoprotein-41 TM domain (SEQ ID NO: 6) and the tPA signal peptide can be fused to the N-terminus of the FL-chimeric protein (SEQ ID NO: 9). An alternative approach is to produce a glycosyl-phosphatidylinositol (GPI)-anchored form the FL adjuvant molecule (designated as FL-GPI) using a pcDNA3-GPt cassette (GenBank access number x52645), which was previously used to produce GM-CSF in an active membrane-bound form (Poloso, N.J., et al., Mol. Immunol., 38:803-816, (2002)). GPI-anchored proteins preferentially associate with lipid rafts, which are used as sites for virus assembly (Nguyen, D. H. and J. E. Hildreth; J. Virol., 74:3265-3272, (2000)). These chimeric FL constructs can be cloned into pc/pS1 and used to produce rBVs expressing FL fusion proteins.

VEE is a member of the family Togaviridae and is typically transmitted by mosquitoes to humans or other animals, in which it causes fever and encephalitis. Following inoculation into the footpad of mice, the virus was observed to be rapidly transported to the draining lymph nodes. Recent studies have shown that dendritic cells in the lymph nodes are the primary target of VEE infection, and VEE replicon particles were observed to be localized in Langerhans cells, dendritic cells of the skin, following subcutaneous inoculation (Macdonald, G. H., and Johnston, R. E., J Virol., 74(2), 914-22, (2000)). These investigators also showed that the targeting of VEE adjuvant molecules to DCs was dependent upon the specific amino acid sequence of the viral envelope glycoprotein E2. Therefore, virosomes incorporating VEE adjuvant molecules may be used to target dendritic cells.

Dendritic cells use the mannose receptor (MR) as the major receptor for endocytosis of antigens (Sallusto, F., et al., J. Exp. Med., 192(2), 389-400, (1995)). This receptor is a 175 kD protein containing eight carbohydrate recognition domains with high affinity for mannose-rich glycoproteins (Stahl, P. D., Curr Opin Immunol., 4(1), 49-52, (1992) and Ezekowitz, R. A., et al., J Exp. Med., 172(6), 1785-94, (1990)). Following endocytosis, the MR releases its ligand at low pH and it recycles to the cell surface, thus having the capacity to interact with ligands in multiple rounds (Stahl, P., et al.; Cell, 19(1), 207-15, (1980)). It has been suggested that the MR may provide a mechanism for distinguishing self from non-self antigens on the basis of glycosylation patterns since, in higher eukaryotes, mannose residues are usually buried within the carbohydrate moieties of envelope glycoproteins and therefore not available for binding to MR (Sallusto, F., et al., J. Exp. Med. 192(2), 389-400, (1995)). Thus, it may be possible to target virosomes to dendritic cells on the basis of distinct oligosaccharide profiles.

Once dendritic cells take up antigens, immature dendritic cells need to differentiate into professional antigen presenting cells in response to maturation signals. As dendritic cells mature, expression of co-stimulatory molecules and MHC-peptide complexes increase and cytokines are produced (Banchereau, J. & I Steinman, R. M., Nature, 392, 245-52, (1998) and Pierre, P., Turley, et al., Nature, 388, 787-92, (1997)). Interaction between CD40 expressed on antigen presenting cells including dendritic cells and CD40L on activated Th cells is important for T cell dependent B cell activation and isotype switching (Rousset, F., et al, J. Exp. Med., 173, 705-10, (1991)). CD40 ligation with a cell line expressing CD40L activates Langerhans cell-derived dendritic cells, and induces high level expression of MHC II and accessory molecules such as CD80 and CD86 (Caux, C., et al., J. Exp. Med., 180, 1263-1272, (1994)). Cross-linking CD40 with anti-CD40 antibodies plays a role in ablating the tolerogenic potential of lymphoid dendritic cells (Grohmann U., et al., J. Immunol. 166, 277-83, (2001)). It is also shown that signaling through CD40 on the antigen presenting cells can replace the requirement for “help” from CD4 Th cells in inducing CTL activities (Bennett, S. R., et al., Nature, 393, 478-480, 1993 and Schoenberger, S. P., et al., Nature, 393, 480-483, (1998)). In anti-tumor pre-clinical model studies, it is indicated that the main mediator for dendritic cell activation is CD40 receptor engagement (Ribas, A., et al., Cancer Res., 61, 8787-8793, (2001) and Ridge, J. P., et al., Nature, 393, 474-478, (1998)). These studies suggest that CD40L seem to provide important maturation signals for dendritic cells. Therefore, virosomes incorporating CD40L adjuvant molecules may be used to target dendritic cells.

Follicular dendritic cells (FDCs) play an important role in germinal centers, where antibody-forming cells are generated. Recent studies have indicated that FDCs play an important co-stimulatory role in the enhancement of antibody responses (Qin, D., et al. J. Immunol., 161, 4549-4554, (1998); Fearon, D. T. and Carroll, M. C.; Annu. Rev. Immunol., 18, 393-422, (2000); Fakher, M., et al., Eur. J. Immunol., 31, 176-185, (2001) and Tew, J. G., et al., Trends Immunol., 22, 361-367, (2001)). During HIV infection, immune complexes containing virions are found in association with FDCs (Hlavacek, W. S., et al.; Philos. Trans. R. Soc. Lond B Biol. Sci., 355, 1051-1058, (2000); Rosenberg, Y. J., et al., Dev. Immunol., 6, 61-70, (1998); Smith, B. A. et al.; J. Immunol., 166, 690-696, (2001)), and such complexes could play a significant role in effective antigen presentation to B cells for induction of neutralizing antibody as observed during HIV infection in vivo. Because of their close similarity to virions, virosomes may mimic such immune complexes much more closely than soluble antigens.

The FDCs interact with components of the complement system including C3d, and it was recently demonstrated that recombinant proteins containing a segment of the C3d adjuvant molecule (amino acids 1024 to 1320 of SEQ ID NO: 11) fused to an antigen resulted in a striking increase in the efficiency of the antibody response (Dempsey, P. W., et al., Science, 271, 348-350, (1996)). Complement is a plasma protein system of innate immunity that is activated by microorganisms in the absence of antibody (Fearon, D. T. and Austen, K. F., N. Engl. J. Med., 303, 259-263, (1980)). Upon activation, C3d fragment binds to its receptor, CR2 (CD21) which is primarily expressed on B cells and FDCs (Fearon, D. T. and Carter, R. H.; Annu. Rev. Immunol., 13:17-149, (1995)). The presence of C3d adjuvant molecules on the surfaces of the virosomes may result in their enhanced interaction with FDCs and B cells and, thus, stimulation of the antibody responses to viral surface envelope glycoproteins contained in the virosome structure.

Because of the relatively large size of the C3d adjuvant molecule fragment, which is about 300 amino acids in length, two factors may affect its function: 1) its proper exposure for interaction with CR2 on FDC, and 2) its potential interference with the proper folding of the protein antigen. Two alternative approaches can be used to incorporate the C3d fragment into virosomes in order to elucidate antibody responses against viral surface glycoproteins incorporated into the virosomes.

First, the C3d adjuvant molecule fragment (amino acids 1024 to 1320 of SEQ ID NO: 11) can be fused to the N-terminus of the selected viral surface envelope glycoprotein, and a signal peptide can be introduced at the N-terminus of the viral surface envelope glycoprotein. Second, a signal peptide, such as the tPA signal peptide (SEQ ID NO: 9), can be fused to the N-terminus of the C3d adjuvant molecule and a membrane anchoring sequence (TM domain of viral glycoproteins, example SIV envelope TM (SEQ ID NO: 6), or the GPI-anchoring sequence (GenBank access number x52645, SEQ ID NO: 10)) can be fused to the C-terminus of the C3d adjuvant molecule.

Virosomes can be produced by in vitro cell culture expression systems such as, but not limited to, recombinant baculovirus expression system (BEVS) (Yamshchikov, G. V., Ritter, G. D., Vey, M., and Compans, R. W. Virology, 214, 50-58, (1995). Assembly of SIV virosomes containing envelope proteins can be performed using expression systems, such as, but not limited to, a baculovirus expression system (Yamshchikov, G. V., Ritter, G. D., Vey, M., and Compans, R. W., Virology, 214, 50-58, (1995)), recombinant poxvirus expression system (MVA) (Wyatt LS, et al., Vaccine, 15, 1451-8, (1996)), recombinant VSV, recombinant adenovirus, and recombinant DNA expression vectors. In some exemplary embodiments, the virosomes are produced using recombinant BEVS and recombinant poxvirus expression systems.

In general, virosomes can be produced by simultaneously introducing into a cell a viral surface envelope glycoprotein expression vector and, optionally, an adjuvant molecule expression vector. The cell produces a lipid vesicle that incorporates the viral surface envelope glycoprotein and/or the adjuvant molecule. The viral surface envelope glycoprotein and/or the adjuvant molecule are expressed on the virosome surface. Thereafter, the cell produces the virosome (e.g., chimeric and/or phenotypically mixed virosomes). The cells can include, but are not limited to, insect cells (e.g., spodopera frugiperda Sf 9 cells and Sf21 cells) and mammalian cells (e.g., EL4 cells and HeLa cells). The elements for expressing the viral surface envelope glycoprotein and adjuvant molecule can also be included together in a single expression vector, or can be included in two or more expression vectors.

In general, the viral surface envelope glycoprotein expression vector can be produced by operably linking a coding sequence for a viral surface envelope glycoprotein of a virus to an appropriate promoter (e.g., early promoter, late promoter, or hybrid late/very late promoter). The viral surface envelope glycoprotein expression vector can be modified to form a viral surface envelope glycoprotein expression construct. Similarly, the adjuvant molecule expression vector can be produced by operably linking a coding sequence for an adjuvant molecule to an appropriate promoter (e.g., early promoter, late promoter, or hybrid late/very late promoter). The adjuvant molecule expression vector can be modified to form an adjuvant molecule expression construct.

In other embodiments, polynucleotide sequences encoding for at least one viral surface envelope glycoprotein, and, optionally, at least one adjuvant molecule can be included in a single expression vector, or in two or more expression vectors. The one or more expression vectors can be introduced into a host cell, and the proteins can be expressed in the cell, whereby the cell forms the virosome. In embodiments, each of the polynucleotide sequences encoding for the viral surface envelope glycoprotein and the adjuvant molecule is operably linked to an appropriate promoter (e.g., a baculovirus promoter, a recombinant Modified Vaccinia Ankara (MVA) promoter, a CMV promoter, an EF promoter, an adenovirus promoter, a recombinant VSV promoter, a recombinant adenovirus promoter, a recombinant alphavirus promoter, a recombinant herpesvirus promoter, a recombinant poxvirus promoter, a recombinant cytomegalovirus promoter, and a recombinant DNA expression vector). Appropriate promoters include, but are not limited to, a constitutive or inducible promoter and/or an early, late or hybrid late/very late promoter.

Additional embodiments also include methods of immunizing a host by expressing at least one viral surface envelope surface glycoprotein and, optionally, at least one adjuvant molecule in one or more host cells (e.g., via use of one or more expression vectors). The at least one viral surface envelope glycoprotein and at least one adjuvant molecule thus expressed by the host cell(s) assemble to form a virosome. The virosome elicits an immune response from the host, thereby providing future protection from infection by a pathogen corresponding to the proteins expressed by the virosome.

In embodiments where the adjuvant molecule is mannose, the adjuvant molecular expression construct is not needed because the mannose molecules can be chemically added to virosomes after the virosomes are produced.

In embodiments of the present disclosure, chimeric viral surface glycoproteins may be useful for increasing the level of incorporation of viral glycoproteins in virosomes for viruses that may naturally have low levels of incorporation. For instance, HIV-1 is known to contain a low level of Env (7-14 trimers per virion). Thus, approximately only 1.5% of total proteins of the virion are Env proteins. Other enveloped viruses incorporate relatively much higher levels of viral glycoproteins into their virions. For example, the type B retrovirus mouse mammary tumor virus (MMTV) contains up to 58% of envelope protein, and the Pichinde virus, an arenavirus, contains up to 27% of envelope glycoprotein.

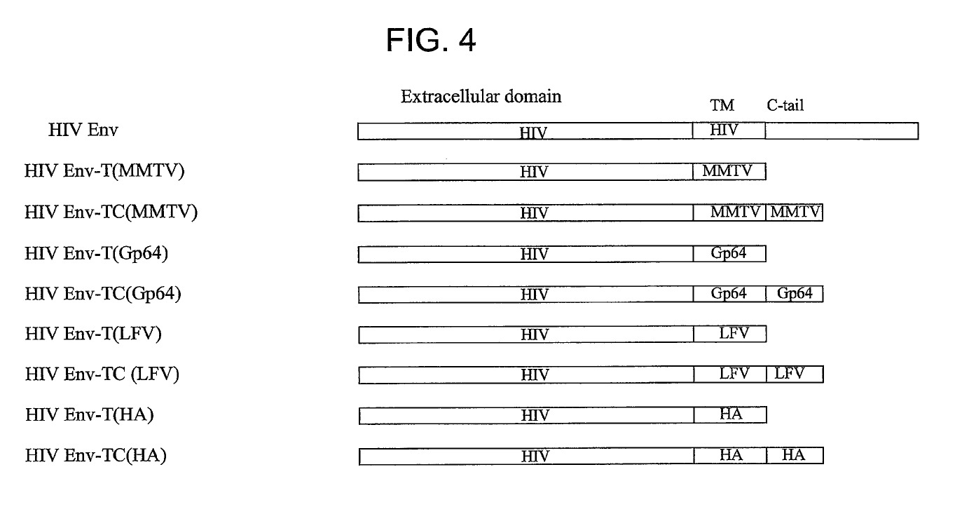

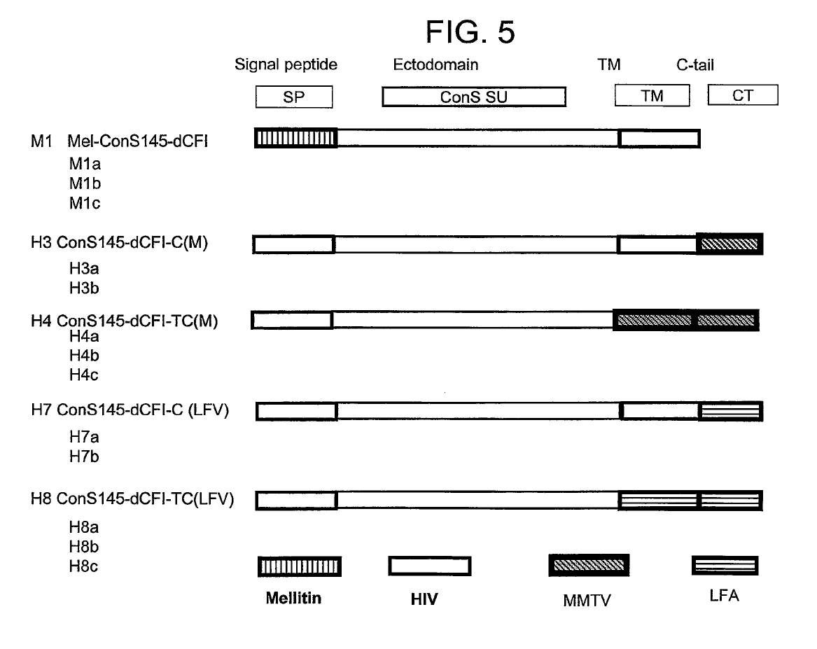

One factor governing the levels of viral envelope protein incorporated into virions may be the transmembrane (TM) and cytoplasmic tail (C-tail) domain. In some embodiments described herein, to enhance the level of HIV Env into virosomes, novel chimeric HIV Env (H3, H4, H7, H8) were constructed by replacing the transmembrane and/or cytoplasmic domains of the HIV envelope glycoprotein with the TM and/or C-tail domains of the mouse mammary tumor virus envelope glycoprotein (MMTV), the baculovirus glycoprotein Gp64 (Gp64), the Lassa Fever virus glycoprotein (LFV), or the influenza surface glycoprotein HA (HA), as illustrated in FIGS. 4 and 5.

Another factor in the level of incorporation of Env proteins in a virion is the signal sequence. The HIV-1 env gene, with its natural signal sequence expressed in any prokaryotic or eukaryotic expression system, showed low levels of synthesis. The signal sequences from honeybee mellitin is known to facilitate the intracellular transport of glycoproteins to the cell surface. Thus, in an embodiment, the natural signal peptide (SP) of HIV-1 Env was replaced with the mellitin SP, as illustrated in FIG. 5 (top) to further enhance the expression of HIV-1 Env on the cell surface, so that more efficient incorporation of HIV Env into virosomes would be achieved.

Virosomes based on cloned viral surface envelope glycoproteins can be readily produced without the expense of undue experimentation by the ordinary skilled artisan using the teachings of the present application taken with vectors as described herein and what is well known to and readily accessible in the art.

In another embodiment, polyclonal and/or monoclonal antibodies capable of specifically binding to the virosome are provided. Antibodies specific for virosomes and viral surface envelope glycoproteins of viruses may be useful, for example, as probes for screening DNA expression libraries or for detecting the presence of the cognate virus in a test sample. Frequently, the polypeptides and antibodies will be labeled by joining, either covalently or noncovalently, a substance that provides a detectable signal. Suitable labels include, but are not limited to, radionuclides, enzymes, substrates, cofactors, inhibitors, fluorescent agents, chemiluminescent agents, magnetic particles and the like. United States Patents describing the use of such labels include, but are not limited to, U.S. Pat. Nos. 3,817,837; 3,850,752; 3,939,350; 3,996,345; 4,277,437; 4,275,149; and 4,366,241, which are hereby incorporated by reference herein.

Antibodies specific for virosomes and retroviral surface envelope glycoproteins may be useful in treating animals, including humans, suffering from cognate viral disease. Such antibodies can be obtained by the methods described above and subsequently screening the viral surface envelope glycoproteins-specific antibodies for their ability to inhibit virus uptake by target cells.

Compositions and immunogenic preparations of the present disclosure, including vaccine compositions, that include the virosomes of the present disclosure and that are capable of inducing protective immunity in a suitably treated host and a suitable carrier therefor are provided. The vaccine preparations of the present disclosure can include an immunogenic amount of one or more virosomes, fragment(s), or subunit(s) thereof. Such vaccines can include one or more viral surface envelope glycoproteins and portions thereof, and adjuvant molecules and portions thereof on the surfaces of the virosomes, or in combination with another proteins or other immunogens, such as one or more additional virus components naturally associated with viral particles or an epitopic peptide derived therefrom. It is preferred for HIV and HTLV, among others, that the route of administration and the immunogenic composition is designed to optimize the immune response on mucosal surfaces, for example, using nasal administration (via an aerosol) of the immunogenic composition.

Immunogenic carriers can be used to enhance the immunogenicity of the virosomes from any of the viruses discussed herein. Such carriers include, but are not limited to, proteins and polysaccharides, microspheres formulated using (e.g., a biodegradable polymer such as DL-lactide-coglycolide, liposomes, and bacterial cells and membranes). Protein carriers may be joined to the proteinases, or peptides derived therefrom, to form fusion proteins by recombinant or synthetic techniques or by chemical coupling. Useful carriers and ways of coupling such carriers to polypeptide antigens are known in the art.

The immunogenic compositions and/or vaccines of the present disclosure may be formulated by any of the methods known in the art. They can be typically prepared as injectables or as formulations for intranasal administration, either as liquid solutions or suspensions. Solid forms suitable for solution in, or suspension in, liquid prior to injection or other administration may also be prepared. The preparation may also, for example, be emulsified, or the protein(s)/peptide(s) encapsulated in liposomes.

The active immunogenic ingredients are often mixed with excipients or carriers, which are pharmaceutically acceptable and compatible with the active ingredient. Suitable excipients include but are not limited to water, saline, dextrose, glycerol, ethanol, or the like and combinations thereof. The concentration of the immunogenic polypeptide in injectable, aerosol or nasal formulations is usually in the range of about 0.2 to 5 mg/ml. Similar dosages can be administered to other mucosal surfaces.

In addition, if desired, the vaccines may contain minor amounts of auxiliary substances such as wetting or emulsifying agents, pH buffering agents, and/or other agents, which enhance the effectiveness of the vaccine. Examples of agents which may be effective include, but are not limited to: aluminum hydroxide; N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE); and RIBI, which contains three components extracted from bacteria: monophosphoryl lipid A, trehalose dimycolate and cell wall skeleton (MPL+TDM+CWS) in a 2% squalene/Tween 80 emulsion. The effectiveness of the auxiliary substances may be determined by measuring the amount of antibodies (especially IgG, IgM or IgA) directed against the immunogen resulting from administration of the immunogen in vaccines which comprise the adjuvant in question. Additional formulations and modes of administration may also be used.

The immunogenic compositions and/or vaccines of the present disclosure can be administered in a manner compatible with the dosage formulation, and in such amount and manner as will be prophylactically and/or therapeutically effective, according to what is known to the art. The quantity to be administered, which is generally in the range of about 1 to 1,000 micrograms of viral surface envelope glycoprotein per dose and/or adjuvant molecule per dose, more generally in the range of about 5 to 500 micrograms of glycoprotein per dose and/or adjuvant molecule per dose, depends on the nature of the antigen and/or adjuvant molecule, subject to be treated, the capacity of the hosts immune system to synthesize antibodies, and the degree of protection desired. Precise amounts of the active ingredient required to be administered may depend on the judgment of the physician or veterinarian and may be peculiar to each individual, but such a determination is within the skill of such a practitioner.

The vaccine or immunogenic composition may be given in a single dose; two dose schedule, for example two to eight weeks apart; or a multiple dose schedule. A multiple dose schedule is one in which a primary course of vaccination may include 1 to 10 or more separate doses, followed by other doses administered at subsequent time intervals as required to maintain and/or reinforce the immune response (e.g., at 1 to 4 months for a second dose, and if needed, a subsequent dose(s) after several months). Preferably, humans (or other animals) immunized with the virosomes of the present disclosure are protected from infection by the cognate virus.

It should also be noted that the vaccine or immunogenic composition can be used to boost the immunization of a host having been previously treated with a different vaccine such as, but not limited to, a DNA vaccine and a recombinant virus vaccine.

Except as noted hereafter, standard techniques for peptide synthesis, cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y., Old Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering: Principles and Methods, Vols. 1-4, Plenum Press, N.Y.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure and the present disclosure and protected by the following claims.

### EXAMPLES

Now having described the virosomes of the present disclosure in general, the examples below describe some embodiments of the virosomes. While embodiments of virosomes are described in connection with the following examples and the corresponding text and figures, there is no intent to limit embodiments of the virosomes to these descriptions. On the contrary, the intent is to cover all alternatives, modifications, and equivalents included within the spirit and scope of embodiments of the present disclosure.

### Example 1

### Production and Characterization of Chimeric RVFV GP Virosomes and GP-SIV Gag Virus Like Particles (VLPs)

Rift Valley Fever Virus (RVFV) is a member of phlebovirus genus within the family Bunyaviridae. It is an enveloped virus with a segmented negative-strand RNA genome consists of three segments: L, M and S. It codes 6 proteins: GN, GC, NC, NS1, NS2 and polymerase. RVFV causes hemorrhagic fever with about 1% fatality. However, it can be aerosolized for infection of both humans and domestic livestock and potentially become endemic if introduced into North America. The present example investigates the production of RVFV virosomes and virus-like particles (VLPs) for vaccine development against RVFV infection. Recombinant baculoviruses expressing the RVFV GN and GC glycoproteins (rBV-GN and rBV-GC) were generated. Co-infection of Sf9 insect cells with rBV-GN and rBV-GC led to release of particle-like vesicles (designated as GP-virosomese or vesicles) containing RVFV glycoproteins.

The genes for RVFV glycoprotein GN and GC subunits were cloned into the recombinant baculovirus (rBV) transfer vector pC/pS1 under a pPol/pCap hybrid promoter, and rBVs expressing RVFV GN (rBV-GN), and GC (rBV-GC) proteins were generated following established procedures. Expression of GN and GC proteins by rBVs in Sf9 insect cells was verified by radioactive labeling coupled with immunoprecipitation using a hyper-immune mouse sera against RVFV.

To determine whether the RVFV glycoproteins can be incorporated into chimeric virosomes and SIV Gag VLPs, Sf9 cells were infected by rBV-GN and rBV-GC at an MOI of 5 for each rBV, and for VLPs were infected along with rBV-SIV Gag at an MOI of 2. At 60 hs post infection, the cell culture medium was collected and clarified by centrifugation at 6000 rpm for 20 min in a Sorvall SS34 rotor, and the virosomes and/or VLPs released into the medium were concentrated by centrifugation at 28000 rpm in a Beckman SW28 rotor. The pellet was resuspended in PBS and the virosomes and/or VLPs were purified by centrifugation through a sucrose gradient (10-50%) at 30000 rpm in a Beckman SW41 rotor at 4° C. for 2 hours. The band containing VLPs or virosomes (easily visible) was collected, diluted in PBS, and VLPs and virosomes were pelleted by centrifugation at 30000 rpm and resuspended in PBS. Protein concentration in the VLP and/or virosome preparation was determined and the VLPs/virosomes were adjusted to a final protein concentration of 1 mg/ml. The VLP/virosome preparations were stored in aliquots at −80° C. and used for VLP/virosome characterization as well as immunization studies as described below.

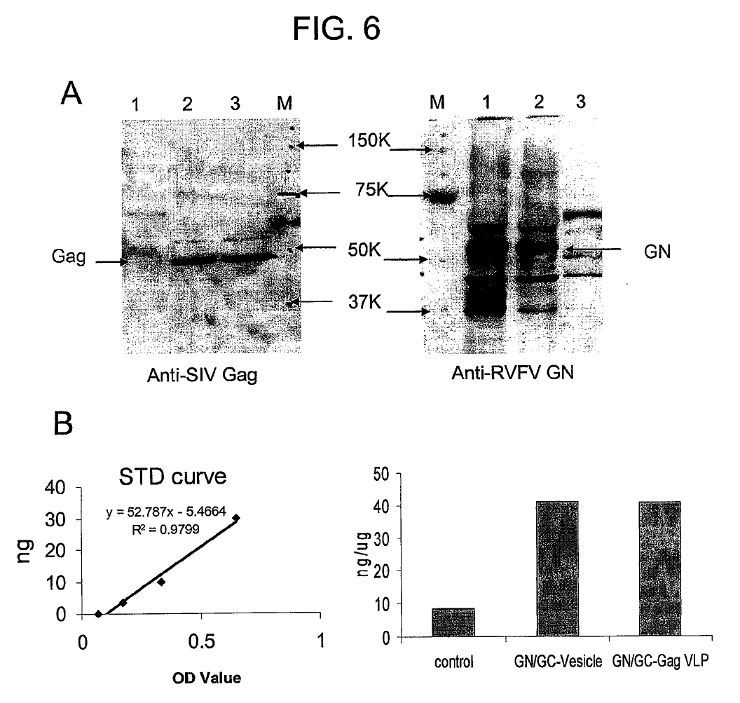

The presence of SIV Gag and RVFV glycoproteins in VLP/virosome preparations were analyzed by SDS-PAGE (5 μg per lane) followed by Western blot using antibodies against SIV Gag or a mixture of monoclonal antibodies against the RVFV GN protein respectively. As shown in FIG. 6A below, the RVFV GN proteins were detected in virosomes produced in rBV-GN and rBV-GC infected Sf9 cells (lane 1), and the SIV Gag proteins were detected in VLPs produced in rBV-SIV Gag infected Sf9 cells (lane 3), while both the RVFV GN and the SIV Gag proteins were detected in VLPs produced in Sf9 cells infected with rBV-SIV Gag plus rBV-GN and rBV-GC (lane 2). The amount of RVFV glycoproteins in virosome and VLP preparations was determined by ELISA. Recombinant vaccinia virus expressing RVFV GN-Histag in which a Histag (6-His) was fused to the C-terminus of the GN extracellular domain and the GN-Histag proteins were expressed in HeLa cells and purified using a histag-protein purification kit (Qiagen). A standard curve was constructed by coating the microtiter plate with serial dilutions of purified GN-Histag proteins mixed with the control SIV Gag VLPs. As shown in FIG. 6B, the amounts of RVFV glycoproteins in GP virosomes and in GP-SIV-Gag VLP preparations are similar to each other, with a calculated concentration of about 40 ng/ug in each preparation.

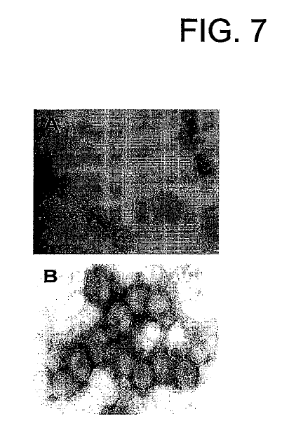

To investigate whether the RVFV glycoproteins are released in the form of virosomes or VLPs, the GP-VLP virosome preparations and GP-SIV-Gag VLP preparations were examined by electron microscopy. As shown in FIG. 7A, particle-like vesicles with less defined membranes were observed in the RVFV GP preparations demonstrating the formation of RVFV GP virosomes. VLPs with uniform morphology were observed in the chimeric GP-SIV-Gag preparations (FIG. 7B), which are similar in size and morphology to the control SIV Gag VLPs.

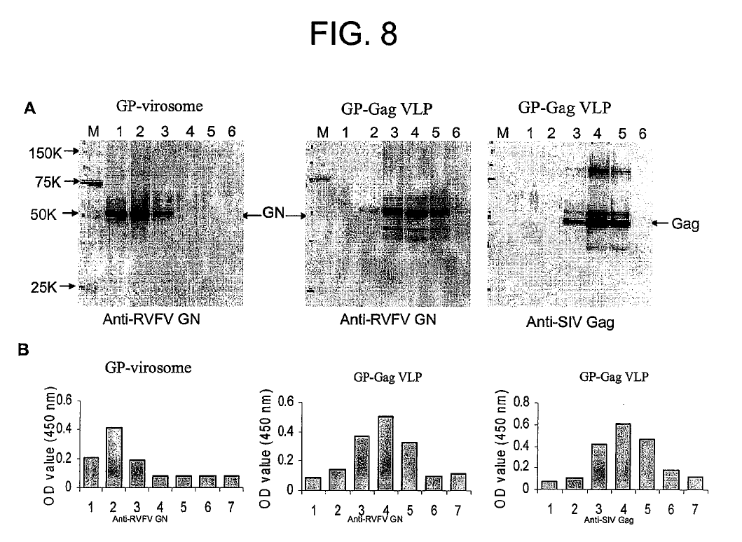

To determine the levels of RVFV glycoproteins associated with the GP virosomes or SIV Gag VLPs in the GP-SIV Gag VLP and GP virosome preparations, the GP virosomes and GP-SIV-Gag VLPs were fractionated by centrifugation through a discontinuous sucrose gradient, and the presence of RVFV glycoproteins as well as SIV Gag proteins in different sucrose fractions were determined by Western blot and ELISA. As shown in FIG. 8, after centrifugation through a sucrose gradient, The RVFV glycoproteins in the GP virosomes localized mainly in the top fractions (fractions 1 to 3) and the majority of RVFV glycoproteins in GP-SIV Gag VLPs co-localized with the SIV Gag proteins in the middle fractions (fractions 3 to 5, 30-50%). These results show that a significant amount of RVFV glycoproteins are incorporated into the virosomes or the SIV Gag VLPs.

To determine their potential for use as a vaccine antigen, the immunogenicity of the virosomes and VLPs were evaluated in mice. Balb/c mice (groups of 6) were immunized by intramuscular injection with GP virosomes or GP-Gag VLPs, each containing 2 ug of RVFV glycoproteins (about 50 ug total protein as determined by ELISA). The control group mice were immunized with 50 ug of SIV Gag VLPs. Mice were boosted at 3 and 6 weeks after priming with the same virosome or VLP preparations at the same dose. Blood was collected by retro-orbital bleeding at two weeks after each immunization and serum samples were heat-inactivated and stored at −80° C. until analysis.

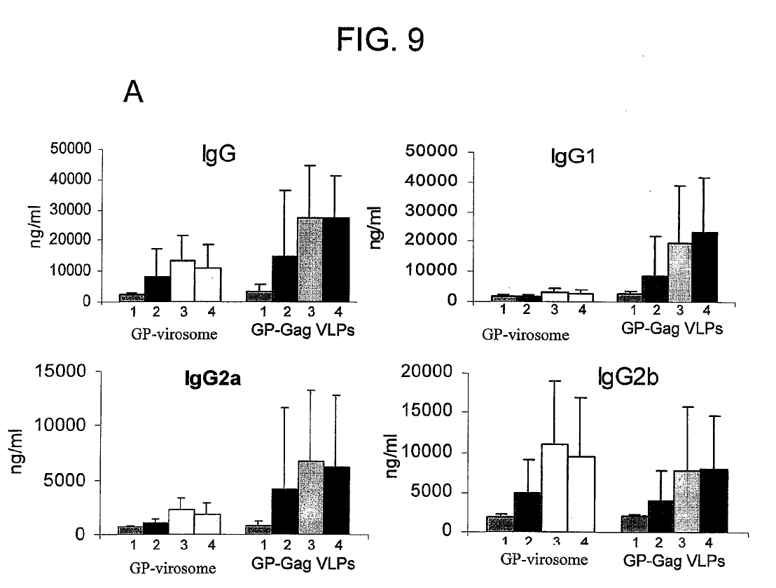

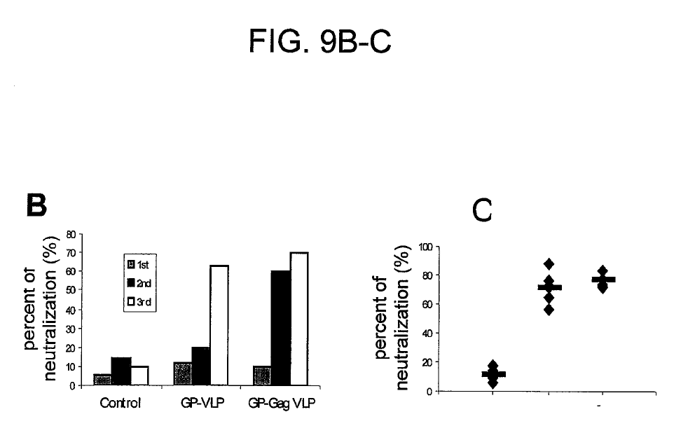

The levels of antibodies specific for the RVFV glycoprotein GN in mouse sera were determined by ELISA using purified GN-Histag proteins as coating antigens. As shown in FIG. 9A, immunization with GP virosomes or GP-Gag VLPs induced significant levels of antibodies against the RVFV GN. The neutralizing activity of sera samples from immunized mice against RVFV were determined by a neutralization assay against RVFV MP12 (obtained from the US Army Medical Research Institute), an attenuated RVFV strain that can be used under BSL-2 containment. MP12 was grown in VERO E6 cells, and virus released into the medium was harvested 72 hr post infection, clarified of cell debris by centrifugation at 1200 rpm for 10 min, and stored at −80° C. in aliquots. The titers of MP12 virus stocks were determined by a plaque assay.

Briefly, serial 10-fold dilutions of thawed MP12 stock were added to Vero E6 cells seeded in a 12-well plate in duplicate. After 1 hr incubation at 37° C., unbound viruses were washed off with DMEM, and the cells were overlaid with white agar (1% white agar prepared in DMEM plus 2% fetal calf serum). After 48 hr incubation at 37° C., the cells were overlaid with red agar (1% white agar containing 1% neutral red and no serum), and the number of plaques was counted 12 hr later to determine the virus titer (pfu/ml). Typically, a titer of 2 to 5×10⁸ pfu/ml was obtained.

To assess the neutralizing activity of serum samples from immunized mice, MP12 (100 pfu) was added to sera samples diluted in DMEM (at 1:40 dilution) in duplicates. Virus added to DMEM without serum was used as control. After 1 hr incubation at 37° C., the virus-serum mixture (in serial 3-fold dilutions) was added to VERO E6 cells seeded in a 12-well plate to determine the residual virus titer by plaque assay. The neutralizing activity of sera samples was calculated as the percentage of plaque number reduction in comparison to control wells (virus incubated with DMEM only) using the formula: (the average number of pfu in control well—the average plaque of pfu in sample well)/the average number of pfu in control well×100%. As shown in FIG. 9B, pooled serum samples from mice immunized with GP virosomes and GP-Gag VLPs exhibited similar levels of neutralizing activity after the third immunization. Also, as shown in FIG. 9C, the neutralizing activity of individual sera samples collected from each group showed that the serum samples from GP virosome and GP-Gag VLP immunized mice exhibit significantly higher levels of neutralizing activity than the serum samples from the control SIV Gag-VLP immunized mice.

In summary, expression of RVFV glycoproteins in Sf9 insect cells by recombinant baculovirus led to the release of RVFV glycoproteins associated with particle-like vesicles (GP virosomes) and co-expression of RVFV glycoprotiens with the SIV Gag protein led to the release of chimeric VLPs containing RVFV glycoproteins (GP-SIV Gag VLPS). Both GP preparations were found to induce high levels of antibodies against RVFV glycoproteins that neutralize RVFV infectivity. These results demonstrate the potential of virosomes and VLPs containing RVFV glycoproteins for the development of an effective vaccine.

### Example 2

### Virosomes with Chimeric RVFV GN and GC Proteins with the MuLV Cytoplasmic Tail

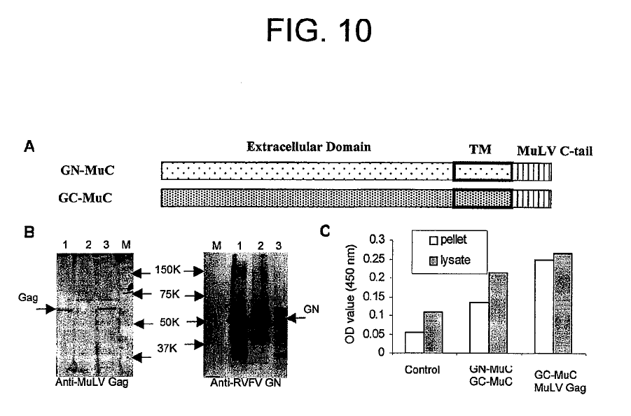

This example illustrates the release of RVFV glycoproteins in virosomes and Virus Like Particles (VLPs). Chimeric RVFV GN and GC proteins containing the MuLV Env cytoplasmic tail were made and expressed in virosomes or in VLPs by co-expression of MuLV Gag proteins. For the VLPs, the gene of the MuLV Gag protein (Moloney strain) was cloned into the rBV transfer vector pC/pS1, and rBV -MuLV Gag was generated following established protocols. To enhance the levels of RVFV glycoprotein incorporation into the MuLV Gag VLPs and the virosomes, genes for chimeric GN and GC proteins were constructed by replacing their cytoplasmic tail with the cytoplasmic tail of the MuLV Env protein, designated as GN-MuC and GC-MuC respectively (FIG. 10A), and rBVs expressing these chimeric proteins were generated.

Expression and release of MuLV Gag and chimeric GN-MuC and GC-MuC proteins in Sf9 cells by recombinant baculoviruses were analyzed by Western blot and ELISA using antibodies against MuLV Gag or RVFV glycoproteins. Sf9 cells (10-7) were infected by rBV-GN-MuC and rBV-GC-MuC at the MOI of 5 with (VLPs) or without (virosomes) rBV-MuLV Gag at the MOI of 2 as indicated. Sf9 cells infected by rBV-MuLV Gag at the MOI of 2 were used as the control. At 60 hr post infection, the cell medium was collected, clarified of cell debris by centrifugation at 1200 rpm for 10 min, and the proteins in the medium were pelleted by centrifugation at 35000 rpm in a SW41 rotor then dissolved in 60 ul of lysis buffer. The GN-MuC and GC-MuC chimeric proteins and the MuLV Gag protein released into the medium were detected by SDS-PAGE (10 ul per lane) followed by Western blot using antibodies against the MuLV Gag protein or monoclonal antibodies against the RVFV GN protein. As shown in FIG. 10B, the MuLV Gag protein (65 Kd in molecular weight) is released into the medium from Sf9 cells infected with rBV-MuLV Gag alone (lane 3) or together with rBV-GN-MuC and rBV-GC-MuC (lane 1). Similarly, the GN-MuC and GC-MuC chimeric proteins were also released into the medium when expressed with the MuLV Gag (lane 1, VLPS) or without the MuLV Gag (lane 2, virosomes).

The levels of chimeric GN-MuC and GC-MuC proteins in cell lysate and in the medium were compared by ELISA. The infected Sf9 cells were lysed by 100 ul of lysis buffer. The cell lysates and the lysed pellets were coated onto a Microtiter plate (10 ul per well) and the levels of RVFV glycoproteins in cell lysates and released protein pellets were compared by ELISA using mouse sera against RVFV as the primary antibody and HRP-conjugated Rabbit-anti-mouse IgG as the secondary antibody. As shown in FIG. 10C, similar levels of chimeric RVFV glycoproteins (GN-MuC and GC-MuC) were expressed in Sf9 cells infected with rBV-GN-MuC and rBV-GC-MuC (virosomes) or with rBV-GN-MuC and rBV-GC-MuC plus rBV-MuLV Gag (VLPs).

### Example 3

### Ebola Virosomes and VLPs Produced in Insect Cells Exhibit Dendritic Cell Stimulating Activity and Induce Neutralizing Antibodies

Viruses of four distinct families, arenaviruses, filoviruses, bunyaviruses, and flaviviruses have been found to cause viral hemorrhagic fevers (VHFs), a group of diseases with common symptoms including fever, fatigue, and muscle ache in mild cases and hemorrhage, shock, coma, seizures, and death in severe cases. Ebola virus is a member of the filoviridae family, and causes severe hemorrhagic fevers in humans and primates with a mortality rate up to 90%, for which no effective treatment or vaccine is available at present. The current outbreak of Marburg virus, another member of the filoviridae family, which has caused infection of almost 400 people with more than 300 fatalities in Angola, further emphasizes the urgent need to development an effective vaccine strategy against the Ebola virus as well as the closely related Marburg virus. The immune response that can provide an effective protection against Ebola or Marburg virus infection and pathogenesis has not been clearly defined and accumulated evidence suggests that both antibody and cellular immune responses will be required. Although viral vector-based vaccine strategies provides hope for the containment of emergency outbreaks, the pre-existing immune response against these viral vectors may reduce their efficacy in human applications. Moreover, the induction of immune responses against viral vector antigens may limit or preclude the possibility of boosting immunity for obtaining long-lasting protection or the successful vaccination against a different strain should it arise in future. These limitations of viral vector-based vaccines underscore the need for the development of alternative vaccines that can be administered repetitively.

Filoviruses are a family of enveloped, nonsegmented negative-stranded RNA viruses encoding 7 proteins, of which the glycoprotein GP is the only protein that forms the spikes on the surfaces of mature virions and mediates virus entry into the cell. The Ebola GP is a type I transmembrane protein which is cleaved into two subunits (GP1 and GP2) by a cellular protease during transport to the cell surface. Infection of cells by Ebola virus leads to assembly and budding of filamentous virus particles. The Ebola matrix protein VP40 is the most abundant protein in the virion and has been shown to provide the driving force for the formation of filamentous virus particles. Like the matrix proteins or Gag proteins of other enveloped RNA viruses, Ebola VP40 contains late domains containing the PTAP and PPXY motifs, which interact with the WW domains of cellular proteins such as Nedd4 and TSg101 and play critical roles in the assembly of virus particles. Expression of Ebola VP40 alone results in efficient assembly and budding of Ebola virus-like particles (VLPs) from mammalian cells. Furthermore, co-expression of Ebola VP40 and GP proteins results in release of Ebola VLPs in mammalian cell lines. However, expression of GP alone also results in release of pleiomorphic vesicles containing GP spikes on their surfaces (virosomes). VLPs for both Ebola and Marburg viruses produced in mammalian cells can confer effective protections against lethal challenges in animal models, indicating that the VLPs represent a promising vaccine strategy for the control and prevention of filovirus infection and associated hemorrhagic fevers.

VLPs of several viruses have been successfully produced from insect cells using recombinant baculoviruses, and these VLPs induce both antibody and cellular immune responses when used to immunize animals. This example investigated the assembly and release of Ebola VLPs and virosomes from insect cells using recombinant baculoviruses expressing GP proteins or co-expressing VP40 and GP proteins, respectively, and characterized the antibody responses induced by these virosome and VLP preparations in mice. The results show that Ebola virosomes and VLPs produced in insect cells exhibit the ability to stimulate cytokine secretion from dendritic cells, and immunization with these Ebola virosomes and VLPs can induce neutralizing antibodies against the Ebola GP protein. Production of virosomes and VLPs in insect cells gives high yields and can be easily adapted for large-scale production, thus representing an attractive approach for the development of an effective vaccine against Ebola virus infection.

### Results

### Expression of VP40 in Insect Cells Leads to Budding and Release of EBOV VLPs.

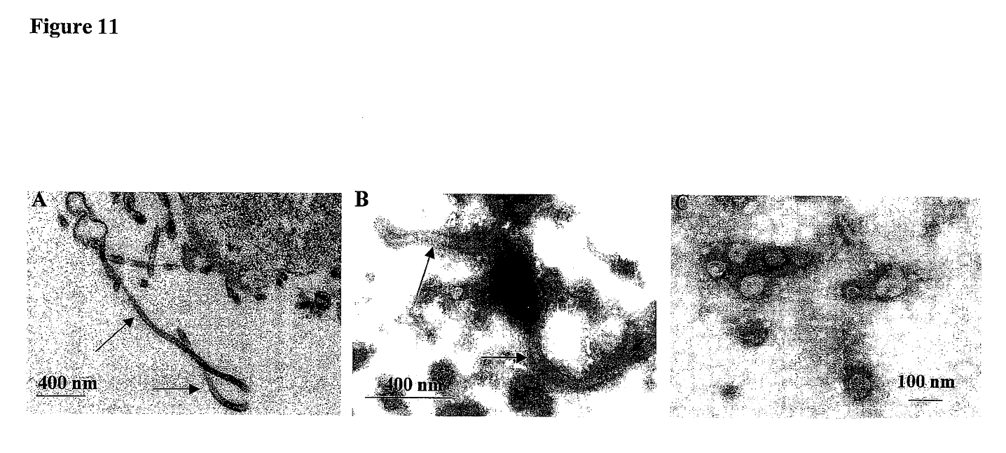

Expression of HIV or SIV Gag proteins in insect cells by infection with recombinant baculoviruses leads to assembly and release of VLPs that are morphologically similar to those observed in mammalian cells. To investigate whether expression of VP40 in insect cells can also lead to assembly of Ebola VLPs, Sf9 insect cells were infected with rBV-VP40 at an MOI of 2 and at 24 hr post infection, the cells were fixed, sectioned, and examined under a transmission electron microscope. As shown in FIG. 11A, infection of Sf9 cells with rBV-VP40 led to formation and budding of filamentous Ebola VLPs from the cell surface as detected by electron microscopy. These filamentous VLPs are about 70 nm in diameter and 800 to 1500 nm in length, which are similar in size and morphology to the virus particles observed in Ebola virus infected cells, indicating that the VP40 protein alone is sufficient for assembly of VLPs when expressed in insect cells. Furthermore, as shown in FIG. 11B, filamentous virus-like particles were observed in the VLP preparations from SF9 cells infected with rBV-VP40 and rBV-GP, similar to those observed budding from the cells (FIG. 11A). Similar filamentous particles were also observed in the VLP preparations from the supernatant of Sf9 cells infected with rBV-VP40 alone (not shown). On the other hand, pleiomorphic vesicles (rBV-GP virosomes) that are 70 to 150 nm in diameter were observed in the medium from Sf9 cells infected with rBV-GP alone (FIG. 11C).

### Production and Characterization of Ebola Virosomes and VLPs Produced in Insect Cells

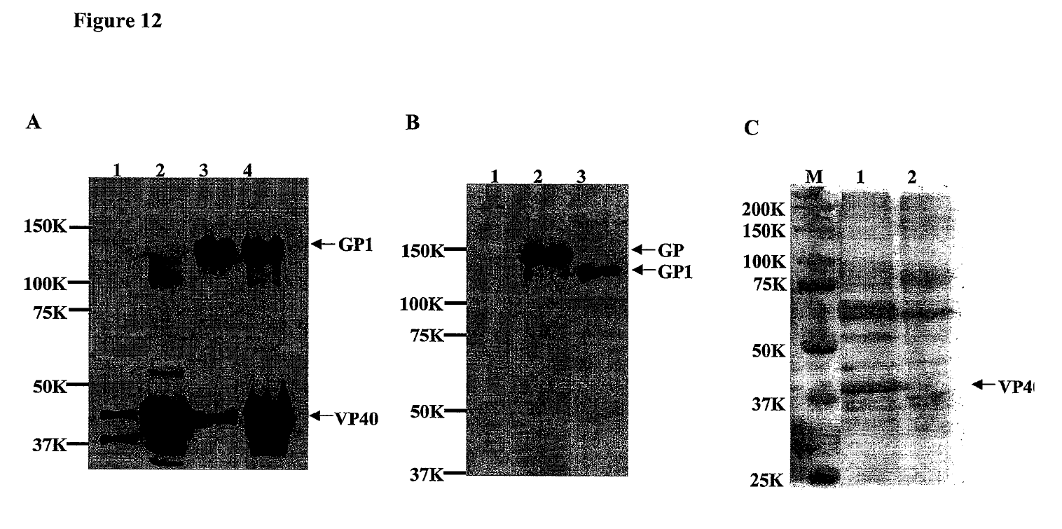

For large-scale production of virosomes and VLPs, Sf9 cells (2×10⁶/ml) were infected with rBV-VP40 alone, rBV-GP alone, or rBV-VP40 and rBV-GP together at an MOI of 2 and 5 respectively, and VLPs were purified in a discontinuous sucrose gradient (10-50%). A visible band between the 30% and 50% sucrose layers was collected, concentrated by ultracentrifugation and then resuspended in PBS giving a final protein concentration of 1 ug/ul. We compared the protein profiles of Ebola VLP preparations from Sf9 cells infected with rBV-VP40 and rBV-GP and the VLP preparations from Sf9 cells infected with rBV-VP40 or the virosome preparations from Sf9 cells infected with rBV-GP alone. VLP preparations from Sf9 cells infected with rBV-Gag (expressing the simian immunodeficiency virus Gag protein) were used as a control. As shown in FIG. 12A, analysis of the protein profile by SDS-PAGE followed by Western blot using a mixture of monoclonal antibodies against VP40 and GP proteins showed that both VP40 and GP are present in the VLP preparations produced by co-infection of Sf9 cells with rBV-VP40 and rBV-GP. As expected, only the VP40 protein or GP protein was detected in the preparations from cells infected with rBV-VP40 or rBV-GP alone respectively.

The Ebola GP protein expressed in mammalian cells is synthesized as a precursor and it is cleaved into two subunits, GP1 and GP2, which are linked by a disulfide bond. The antibody against GP is specific for the GP1 subunit. To investigate whether the GP proteins in the VLP and virosome preparations produced in insect cells are cleaved, the mobility of GP protein in virosome and VLP preparations were compared in the presence or absence of the reducing reagent β-mercaptoethanol. As shown in FIG. 12B, the GP protein exhibited a faster mobility by SDS-PAGE after treatment with β-mercaptoethanol, indicating that it was efficiently cleaved in insect cells. Protein profiles for VP40-GP VLPs and GP virosomes were also examined by SDS-PAGE and coomassie blue staining. As shown in FIG. 12C, VP40 proteins are readily detected in VP40-GP VLPs. However, GP proteins were not detected in either VLP preparation, probably due to the lower relative levels of GP in VLPs and virosomes and/or inefficient staining of glycoproteins by coomassie blue. A protein band about 60 Kd in molecular weight was also detected by coomassie blue staining in both VP40-GP VLPs and GP virosomes, which is probably Baculovirus glycoprotein GP64 based on its apparent molecular weight.

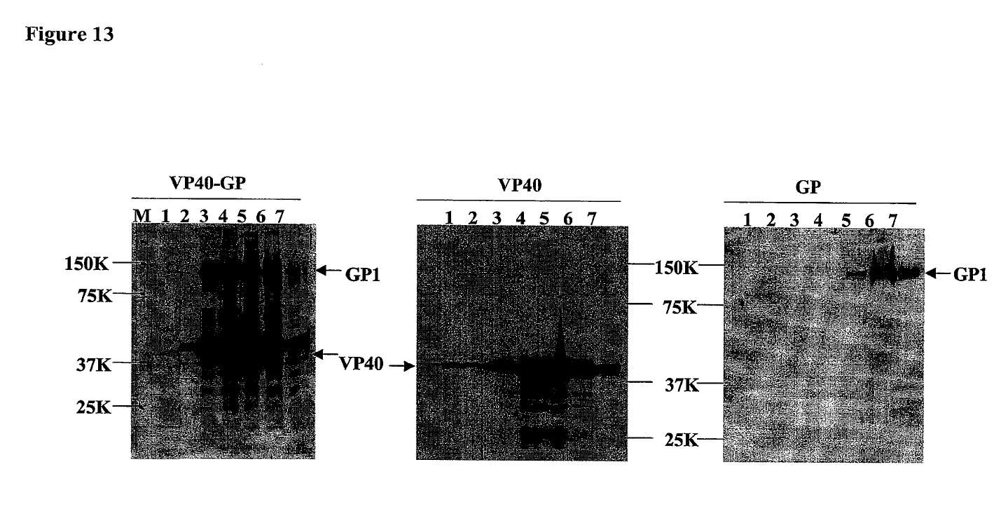

To demonstrate that the GP proteins are incorporated into the VLPs and virosomes, 5 ug of the VLP/virosome preparations from the supernatant of Sf9 cells infected with rBV-VP40 and rBV-GP as well as rBV-VP40 or rBV-GP alone were loaded on top of a discontinuous sucrose gradient (10-50%), followed by centrifugation at 30K RPM for 1 hr in a Beckman SW41 rotor. Fractions were collected and the proteins in these fractions were concentrated and analyzed by SDS-PAGE and Western blot. As shown in FIG. 13, the majority of both VP40 and GP in the VLP preparation from rBV-VP40 and rBV-GP infected cells co-sedimented in fractions 3 to 6. Similarly, the majority of VP40 in the VLP preparation from rBV-VP40 infected cells was also detected in fractions 3 to 6. On the other hand, the majority of GP from the rBV-GP virosome infection was detected in fractions 5 to 7. These result showed that the majority of the released GP proteins in the medium from cells expressing both VP40 and GP proteins co-localized with the VP40 proteins in the sucrose gradient, and sedimented in different fractions compared with the released GP proteins from cells infected with rBV-GP alone, indicating that the GP protein is incorporated into either virosomes (with GP alone) or into VLPs formed by the VP40 proteins.

Stimulation of Cytokine Secretion from Dendritic Cells (DCs) by Ebola Virosomes and VLPs.

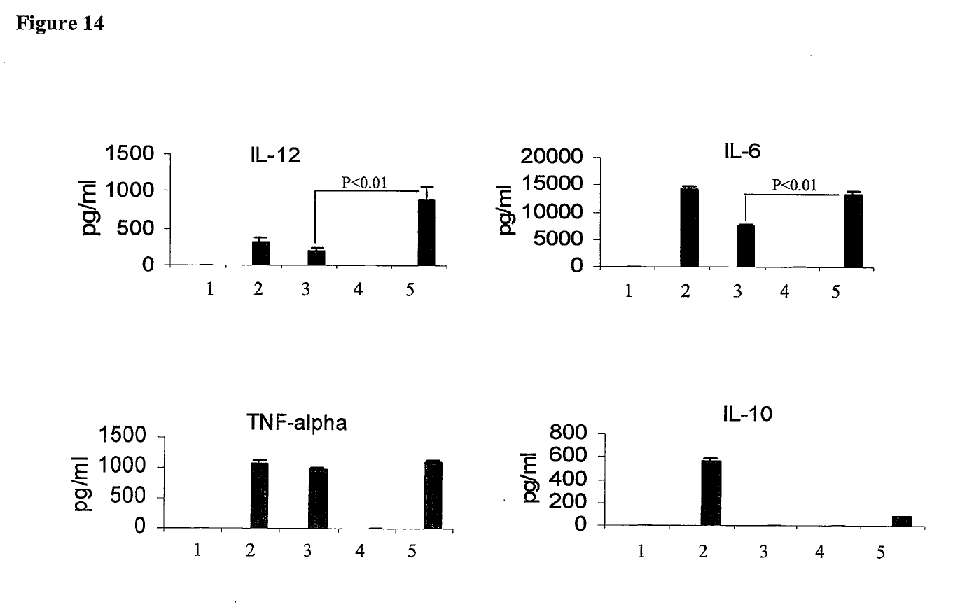

To determine whether Ebola virosomes and VLPs produced in insect cells are able to stimulate cytokine secretion by DCs, DCs were incubated with VP40-GP VLPs, VP40 only VLPs, GP virosome preparations, as well as LPS (positive control) or medium only (negative control) for 24 hrs and the amounts of cytokines secreted into the medium were determined by ELISA. As shown in FIG. 14, incubating DCs with Ebola VP40-GP VLPs induced secretion of cytokine and chemokines such as IL-6, IL10, IL-12 and TNF-alpha. The levels of IL-6 and TNF-alpha induced by VP40-GP VLPs were similar to those induced by LPS, while the level of IL-12 is higher and the level of IL-10 is lower than the levels induced by LPS, respectively. These results indicate that the Ebola VLPs produced in insect cells exhibited the biological activity for DC-stimulation. Furthermore, incubating DCs with preparations from Sf9 cells infected with rBV-GP virosomes (as shown in FIG. 11C) also induced secretion of similar levels of TNF-alpha, as well as inducing secretion of IL-12 and IL-6. On the other hand, incubating DCs with VLPs containing VP40 only did not induce secretion of any cytokines at detectable levels. These results demonstrate that the presence of GP in virosomes or in the VLPs is important for their DC-stimulating activity.

Induction of Antibody Responses by Immunization with Ebola virosomes and VLPs.

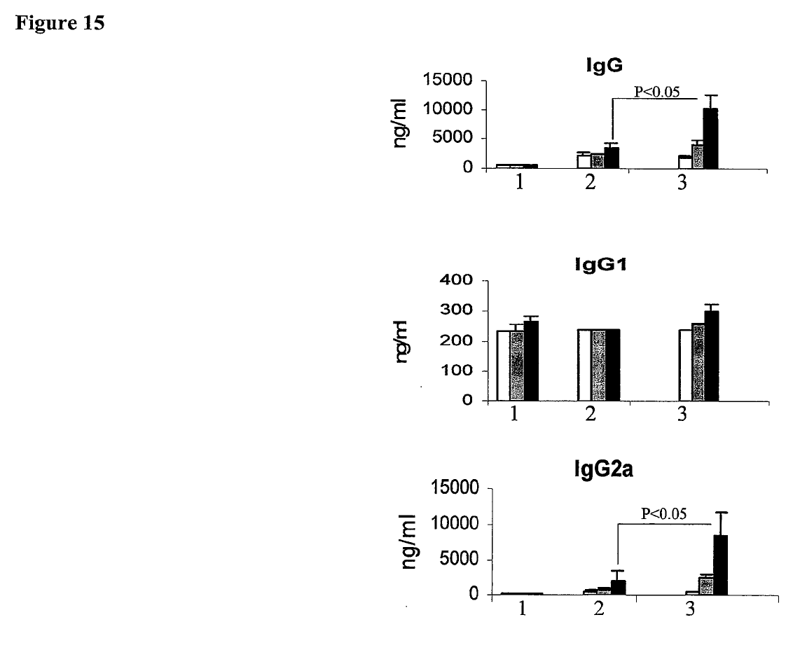

The immunogenicity of the virosomes and VLPs produced in insect cells was evaluated by immunization of mice with the GP virosome preparations or the VP40-GP VLPs. Mice were immunized intramuscularly 3 times at 4-week intervals with 50 ug of Ebola VP40-GP VLPs or with the GP virosomes (dissolved in PBS at 1 ug/ul). As shown in FIG. 12A, the GP virosome preparation contained similar or slightly higher levels of GP proteins than the same amount of VLP preparation. Serum samples were collected at two weeks after each immunization and the levels of antibody responses specific for the Ebola virus GP were determined by ELISA using Ebola GP1-histag fusion proteins purified from HeLa cells as coating antigens. As shown in FIG. 15, significant levels of antibodies against Ebola virus GP1-histag proteins were induced after two immunizations with VP40-GP VLPs, and the levels of antibody response were significantly boosted by the third immunization. Immunization with GP virosome preparations also induced significant levels of antibody response after three immunizations. Moreover, it is interesting to note that most of the Ebola GP-specific antibodies induced by VP40-GP VLPs or GP virosomes were of the IgG2a subtype while the levels of IgG1 antibodies were not significantly higher than those induced in the control group, indicating the induction of a strong Th1-biased immune response.

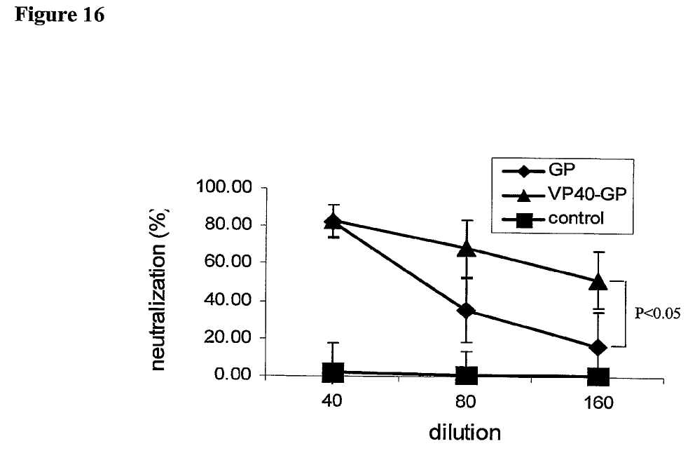

To assess whether the antibodies induced by VP40-GP VLPs or GP virosome preparations can neutralize virus infection, a pseudotype virus-neutralization assay adapted from an HIV neutralization assay system was used as described in Bu et al., 2004 and Ye et al., 2004, which are hereby incorporated by reference herein. Env-deficient HIV virus was pseudotyped with Ebola GP protein by co-transfection of 293T cells with a DNA vector expressing Ebola GP and the HIV cDNA, in which a frameshift deletion mutation was introduced in the env gene. The ability of the Ebola GP to mediate infection of JC53 cells, a cell line expressing β-gal under the control of the HIV LTR (Derdeyn C. A., Decker J. M., Sfakianos J. N., Wu X., O'Brien W. A., Ratner L., Kappes J. C., Shaw G. M., Hunter E., 2000. Sensitivity of human immunodeficiency virus type 1 to the fusion inhibitor T-20 is modulated by coreceptor specificity defined by the V3 loop of gp120 J. Virol., 74, 8358-67; Wei X., Decker J. M., Wang S., Hui H., Kappes J.C., Wu X., Salazar-Gonzalez J. F., Salazar M. G., Kilby J. M., Saag M. S., Komarova N. L., Nowak M. A., Hahn B. H., Kwong P. D., Shaw G. M., 2003. Antibody neutralization and escape by HIV-1. Nature, 422, 307-12, which are hereby incorporated by reference), was determined by counting the number of β-gal-expressing cells. Infection of JC53BL cells by GP-pseudotyped virus was completely blocked by hyper-immune mouse sera against Ebola virus at over 1:1000 dilution (data not shown), indicating that the entry of the pseudotyped viruses into the cells is mediated by the Ebola GP protein. As shown in FIG. 16, sera from both VP40-GP VLP- and GP virosome-immunized mice exhibited a neutralizing activity of about 80% at 1:40 dilution, while sera from the control mice did not show significant levels of neutralizing activity against the pseudotyped virus. These results correlate with the levels of GP-specific antibodies induced by VP40-GP VLPs and GP virosome preparations, and show that Ebola virosomes and VLPs produced in insect cells using the recombinant baculovirus expression system can induce neutralizing antibodies that can prevent Ebola GP protein-mediated virus infection.

### Discussion

In this study, the assembly of Ebola virosomes and VLPs in insect cells was examined using recombinant baculoviruses. The present results show that expression of VP40 alone or together with GP in insect cells led to efficient assembly and release of VLPs, which exhibit a filamentous structure resembling Ebola virions. On the other hand, expression of Ebola GP alone in insect cells led to release of GP virosomes. By co-expression with VP40, GP was found to co-sediment with VP40 in a sucrose gradient, and exhibited a different sedimentation pattern from GP virosomes, indicating that the GP proteins were either incorporated into virosomes or incorporated into the VLPs formed by the VP40 proteins. Moreover, the GP proteins in both the virosomes and VLPs were found to be efficiently processed into the GP1 and GP2 subunits that are linked by disulfide bonds. The yield for purified virosomes and VLPs produced in Sf9 insect cells was typically between 5 to 10 mg per liter of cell culture.

One of the distinctive properties of Ebola virosomes and VLPs is their ability to stimulate DCs or macrophages to secret cytokines. The present results further demonstrated that the VP40-GP virosomes and VLPs produced in insect cells were able to stimulate cytokine secretion by DCs. In contrast, the VP40-VLPs formed by VP40 alone did not stimulate any detectable cytokine secretion, indicating that the presence of GP is critical for the observed cytokine stimulation activity. It is interesting to note that glycoproteins synthesized in insect cells lack modification by complex carbohydrates, and the side-chains are of the high-mannose type. The results of this example suggest that the glycosylation difference between mammalian and insect cells does not significantly affect the ability of Ebola VLPs to stimulate cytokine secretion by DCs, indicating that the Ebola virosomes and VLPs produced in insect cells possess similar functional activities to those produced in mammalian cells. It has been reported that, unlike Ebola VLPs, the purified Ebola GP proteins could not stimulate cytokine secretion by macrophages (Wahl-Jensen V., Kurz S. K., Hazelton P. R., Schnittler H. J., Stroher U., Burton D. R., Feldmann H., 2005. Role of Ebola virus secreted glycoproteins and virus-like particles in activation of human macrophages. J. Virol., 79, 2413-9, incorporated herein by reference). However, the present data indicate that the GP virosome preparations devoid of VP40 were also able to stimulate cytokine secretion by DCs. Taken together, these observations suggest that the presentation of Ebola GP proteins in a membrane bound or multivalent form may be important for exhibiting DC-stimulating activity.

Evaluation of immunogenicities in mice showed that both VP40-GP VLPs and GP virosome preparations induced antibody responses against GP in immunized animals that are mainly of the IgG2a subtype, indicating the induction of strong Th1-biased immune responses. The Ebola GP protein expressed in insect cells has been shown to bind to monoclonal antibodies against different epitopes (Meliquist-Riemenschneider J. L., Garrison A. R., Geisbert J. B., Saikh K. U., Heidebrink K. D., Jahrling P. B., Ulrich R. G., Schmaljohn C. S., 2003. Comparison of the protective efficacy of DNA and baculovirus-derived protein vaccines for EBOLA virus in guinea pigs. Virus Res., 92, 187-93, incorporated herein by reference). We observed that both VP40-GP VLPs and the GP virosome preparations induced neutralizing antibodies that can block virus infection mediated by the Ebola GP protein, indicating that the neutralizing epitopes are preserved in the Ebola GP proteins synthesized in insect cells. In agreement with the GP-specific antibody responses, sera from mice immunized with VP40-GP VLPs and sera from mice immunized with the GP virosome preparations exhibited neutralizing activities.

Virosomes and/or VLPs can be administered repeatedly to vaccinated individuals, and their nonreplicative nature and lack of viral genomic RNA make them safe for broad and repeated application. Thus virosomes and VLPs represent an attractive vaccine strategy against filovirus infection. Production of Ebola virosomes and VLPs in insect cells using recombinant baculoviruses gives high yield, and the production process can be easily adapted for large-scale manufacture, offering an attractive approach for the development of an effective and economical vaccine strategy to prevent Ebola virus infection.

### Materials and Methods

### Cells and Antibodies

Spodoptera frugiperda Sf9 cells were cultured in SF-900 II serum-free medium with penicillin/streptomycin in suspension. Monoclonal antibodies against Ebola VP40 and GP proteins were provided by Dr. Y. Kawaoka.

### Generation of Recombinant Baculoviruses.

Recombinant baculoviruses expressing Ebola VP40 or GP proteins were generated following similar procedures as described in previous studies (Yamshchikov G. V., Ritter G. D., Vey M., Compans R. W., 1995. Assembly of SIV virus-like particles containing envelope proteins using a baculovirus expression system. Virology, 214, 50-8, incorporated herein by reference). The cDNAs for Ebola GP and VP40 proteins (Ebola Zaire strain, Mayinga isolate, provided by Dr. P. Rollin, Center for Disease Control and Prevention) were amplified by PCR and cloned into the plasmid vector pBlueScript IIKS under the T7 promoter, and confirmed by sequencing. These genes were then cloned into an AcMNPV transfer vector Pc/pS1 under the control of a hybrid capsid/polyhedrin promoter (Pcap/polh), and these plasmids were used to generate recombinant baculoviruses expressing Ebola VP40 or GP proteins. Sf9 cells were co-transfected with a mixture of the AcMNPV DNA, and the baculovirus transfer vector pC/pS1 containing Ebola VP40 or GP genes respectively by using a BacGold Cotransfection Kit (Invitrogen) following the manufacturer's instruction. Recombinant baculoviruses were plaque purified and expanded in Sf9 cells to generate virus stocks, and the titers were determined using a Fastplax Assay Kit (Invitrogen).

### Protein Expression and Virosome/VLP Production in Insect Cells.

Sf9 cells in suspension cultures were infected with rBV-VP40 or rBV-GP alone, or co-infected with rBV-VP40 plus rBV-GP at the MOIs (multiplicity of infection) of 2 for rBV-VP40 and 5 for rBV-GP respectively. Media were harvested 48 h post-infection and clarified by centrifugation at 6000 rpm for 30 min. Supernatants were ultracentrifuged at 28000 rpm for 1 hr, and the VLPs in the pellets were resuspended in PBS and further purified on a discontinuous 10-50% (w/v) sucrose gradient. A visible band between the 30% and 50% sucrose layers was collected, concentrated by centrifugation and then resuspended in PBS at a final protein concentration of 1 ug/ul. The presence of VP40 and GP proteins in the purified preparations was analyzed by SDS-PAGE followed by Western blot using antibodies against Ebola viral proteins or coomassie blue staining.

### Examination of EBOV Virosomes and VLPs by Electron Microscopy.

For EM study of virosome and VLP budding from insect cells, Sf9 cells were infected with rBV-VP40 at an MOI of 2. At 24 hr post infection, the cells were fixed with 2% glutaraldehyde and then with 2% osmium tetroxide. Thin-section samples were prepared and examined with a transmission electron microscope. For EM study of purified virosomes and VLPs, preparations from Sf9 cells co-infected with rBV-VP40 plus rBV-GP or with rBV-GP or rBV-VP40 alone were stained with 1% uranyl acetate and then examined under a Hitachi-H7500 transmission electron microscope.

Production of Ebola Virosomes and VLPs in 293T cell.

The genes of Ebola VP40 and GP proteins were cloned into the plasmid vector pcDNA3 under the CMV promoter. 293T cells were seeded in T175 flasks over night and then transfected with pcDNA3-VP40 and pcDNA3-GP using Polyfect (Qiagen) following manufacture's protocols. At 48 hr post transfection, the cell supernatant was collected and virosomes and VLPs were purified by centrifugation through a 20% sucrose cushion at 30K RPM in a Beckman SW41 rotor. The virosomes and VLPs were resuspended in PBS at about 0.5 ug/ul for characterization by Western blot as well as stimulation of DCs.

### Stimulation of Dendritic Cells and Detection of Cytokine Secretion.

Human myeloid dendritic cells were prepared from human PBMC as described in previous studies (Agrawal S., Agrawal A., Doughty B., Gerwitz A., Blenis J., Van Dyke T., Pulendran B., 2003. Cutting edge: different Toll-like receptor agonists instruct dendritic cells to induce distinct Th responses via differential modulation of extracellular signal-regulated kinase-mitogen-activated protein kinase and c-Fos. J. Immunol., 171, 4984-4989; Mahanty S., Hutchinson K., Agarwal S., McRae M., Rollin P. E., Pulendran B., 2003. Cutting edge: impairment of dendritic cells and adaptive immunity by Ebola and Lassa viruses. J. Immunol., 170, 2797-801, incorporated herein by reference). Briefly, PBMC were obtained by aphresis from healthy human donors. Monocyte-derived DC were generated by culturing purified monocytes (Ficoll, and CD14 MACs beads, Miltenyi Biotech) in RPMI 1640 with 10% FBS for 6 days in the presence of GM-CSF (100 ng/ml) and IL-4 (5 ng/ml). This resulted in enrichment for CD11+CD1a+HLA-DR+MDDC (>90%). On day 6 of culture, 0.5×10⁶/ml of immature human myeloid DCs from two different donors were each stimulated in vitro with 10 ug of each respective virosome or VLP preparation as indicated in figure legends (Ebola GP, GP/VP40, VP40), as well as with LPS (10 ng/ml, positive control), or with mock-treated media (negative control) in 12-well plates in duplicates. Supernatants were harvested after 24 hours of at 37° C. in 5% CO₂. Cytokine levels in cell supernatants were measured by ELISA in duplicates using commercially available kits (BD Pharmigen, R&D) for IL-6, IL-8, IL-10, IL-12, as well as TNF-alpha, according to the manufacturer's instruction. The stimulation experiments were repeated once to ensure consistency of obtained results. The results shown represent typical results obtained from two different stimulation experiments and error bars represent standard deviations. Significance of statistical differences in the levels of secreted cytokines stimulated by VP40-GP VLPs or GP virosome preparations were determined by a t test.

### Immunization of Mice and Sample Collection.

Female BALB/c mice (8 weeks old) were obtained from Charles River Laboratory and housed at the Emory University Animal Facility in micro-isolator cages. Groups of mice (six per group) were immunized with a total of 50 μg of indicated purified virosome or VLP preparations per mouse by intra-muscular injection with 25 μl of virosome/VLP preparation in separate sites in both side quadriceps, followed by boosting with the same dose of virosome/VLPs at weeks 4 and 8. Blood samples were collected from the retro-orbital sinus under anesthesia at 1 week prior to the first immunization and 2 weeks after each immunization, and serum samples were collected and stored at −80° C. until further analysis. ELISA.

The coding sequence for the Ebola virus GP1 subunit was linked in frame to the 5′ of a DNA segment encoding for 6 Histidines followed by a stop codon. The gene for the His-tagged GP1 was cloned into the transfer vector pRB21 for generation of recombinant vaccinia viruses expressing the His-tagged Ebola GP1 protein. His-tagged GP1 proteins were expressed in HeLa cells by infection with the recombinant vaccinia virus and purified using Ni-NTA agarose beads (Qiagen). Ebola virus-specific antibodies were measured in individual mouse serum samples by an enzyme-linked immunoabsorbent assay (ELISA) using purified His-tagged GP1 proteins as coating antigens. Briefly, the assays were performed in 96-well polystyrene microtiter plates (Nunc) coated overnight at 4° C. with purified His-tagged GP1 at a concentration of 2 ug/ml. Serial dilutions of serum samples were incubated at RT for 2 hrs on coated and blocked ELISA plates, and the bound immunoglobulins were detected with horseradish peroxidase-labeled goat against mouse IgG, IgG1, or IgG2a (Southern Biotechnology Associates). The wells were developed with TMB (Sigma). The color reaction was stopped with hydrochloric acid (0.2N), and the absorbance at 450 nm was read in an EL312 Bio-Kinetics microplate reader (Bio-Tek Instruments Inc., Winooski, Vt.). A standard curve was constructed by coating each ELISA plate with serial 2-fold dilutions of purified mouse IgG, IgG1, or IgG2a with known concentrations respectively, and the concentrations of Ebola GP-specific antibodies in serum samples were calculated using obtained standard curves and expressed as the amount of antigen-specific antibody in 1 ml of serum samples (ng/ml).

### Neutralization Assay.

Neutralizing antibodies against Ebola GP were analyzed using a single-round infectivity assay adapted from an assay system used in previous studies of HIV (Bu Z., Ye L., Vzorov A., Taylor D., Compans R. W., Yang C., 2004. Enhancement of immunogenicity of an HIV Env DNA vaccine by mutation of the Tyr-based endocytosis motif in the cytoplasmic domain. Virology, 328, 62-73; Ye L., Bu Z., Vzorov A., Taylor D., Compans R. W., Yang C., 2004. Surface stability and immunogenicity of the HIV envelope glycoprotein: The role of the cytoplasmic domain. J. Virol., 78, 13409-13419, incorporated herein by reference). This assay is based on an indicator cell line, JC53-BL, which is a derivative of HeLa cells and contains reporter cassettes of β-galactosidase under an HIV-1 LTR that is activated by expression of the HIV Tat protein (Derdeyn C. A., Decker J. M., Sfakianos J. N., Wu X., O'Brien W. A., Ratner L., Kappes J. C., Shaw G. M., Hunter E., 2000. Sensitivity of human immunodeficiency virus type 1 to the fusion inhibitor T-20 is modulated by coreceptor specificity defined by the V3 loop of gp120. J. Virol., 74, 8358-67; Wei X., Decker J. M., Wang S., Hui H., Kappes J. C., Wu X., Salazar-Gonzalez J. F., Salazar M. G., Kilby J. M., Saag M. S., Komarova N. L., Nowak M. A., Hahn B. H., Kwong P. D., Shaw G. M., 2003. Antibody neutralization and escape by HIV-1. Nature, 422, 307-12, which are hereby incorporated by reference). The Ebola GP pseudotyped HIV was prepared by co-transfection of 293T cells with DNA vectors for the HIV backbone and the Ebola GP, and the titer of the pseudotyped virus was determined in the JC53BL cells. Typically a titer of approximately 10⁵ pfu (blue plaque forming units) per ml was obtained. For analysis of neutralizing antibodies, dilutions of heat-inactivated serum samples were incubated with 100 pfu of pseudotyped virus in a 96-well plate for 1 hr at 37° C., and then added to JC53BL cells in the presence of DEAE-dextran and incubated for 2 days at 37° C. Cells were then stained for β-galactosidase expression. Neutralization was calculated as the percentage of reduction of the number of blue cells in sample wells, which were infected with pseudotyped virus incubated with serum samples, in comparison to the control wells, which were infected with the same dose of pseudotyped virus incubated with the same dilution of group-matched pre-immune serum samples [(the number of blue cells in control wells—the number of blue cells in sample wells)/(the number of blue cells in control wells)×100%].

### Example 4

### Virosomes with Chimeric HIV Envelope Glycoproteins

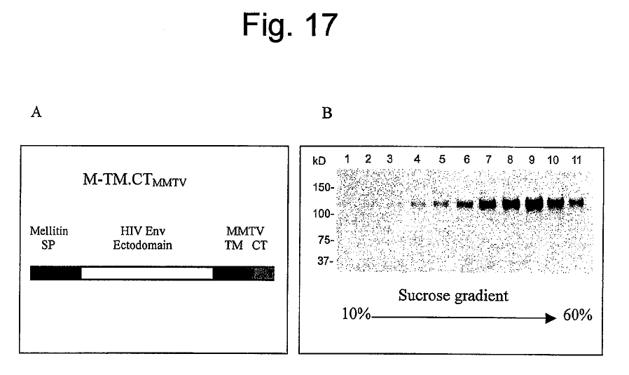

Chimeric HIV envelope glycoproproteins (shown schematically in FIG. 17A) were constructed using the methods described above. In this chimeric HIV env gp construct, M-TM.CT_{MMTV}, the signal peptide (SP) was replaced with that of honeybee mellitin, and the transmembrane (TM) and cytoplasmic (CT) domains were replaced with those of mouse mammary tumor virus (MMTV) Env. Using the methods described in the Examples above, recombinant baculovirus expression systems were used to infect insect to produce virosomes incorporating the chimeric HIV env gp proteins. The HIV Env proteins were found to be incorporated into virosomes at high levels in insect cells infected with recombinant baculoviruses.

These vesicles were separated in a sucrose gradient as described in the Examples above. Western blot analysis of the release of the chimeric M-TM.CT_{MMTV} vesicles after sucrose gradient centrifugation is shown in FIG. 17B. Protein bands were probed with goat anti-gp120 polyclonal antibodies. Lanes 1-11 represent chimeric Env in different fractions of the sucrose gradient from 10 to 60%.

Therefore, having thus described the disclosure, at least the following is claimed:

1. A chimeric virosome, comprising at least one surface molecule selected from the following:
at least one chimeric viral surface envelope glycoprotein;
at least two viral surface envelope glycoproteins expressed on the surface of the virosome, wherein at least one of the viral surface envelope glycoproteins is from a different virus than at least one other viral surface envelope glycoprotein; and
at least one viral surface envelope glycoprotein and at least one adjuvant molecule, wherein at least one viral surface envelope glycoprotein and at least one adjuvant molecule are from different sources.

2. The chimeric virosome of claim 1, wherein the viral surface envelope glycoprotein is selected from: a retrovirus glycoprotein, a bunyavirus glycoprotein, a corona virus glycoprotein, an arenavirus glycoprotein, a filovirus glycoprotein, an influenza virus glycoprotein, a paramyxovirus glycoprotein, a rhabdovirus glycoprotein, an alphavirus glycoprotein, a flavivirus glycoprotein, a herpesvirus glycoprotein, a cytomegalovirus glycoprotein, and combinations thereof.

3. The chimeric virosome of claim 2, wherein the retrovirus glycoprotein is individually selected from: a human immunodeficiency virus (HIV) glycoprotein, a simian immunodeficiency virus (SIV) glycoprotein, a simian-human immunodeficiency virus (SHIV) glycoprotein, a feline immunodeficiency virus (FIV) glycoprotein, a feline leukemia virus glycoprotein, a bovine immunodeficiency virus glycoprotein, a bovine leukemia virus glycoprotein, an equine infectious anemia virus glycoprotein, a human T-cell leukemia virus glycoprotein, a mouse mammary tumor virus envelope glycoprotein (MMTV), and combinations thereof.

4. The chimeric virosome of claim 2, wherein the viral surface envelope surface glycoprotein is individually selected from: a Lassa Fever virus glycoprotein, a Rift Valey Fever Virus (RVFV) glycoprotien, an Ebola Virus glycoprotein, an influenza viral glycoprotein, a Hepatitis C Virus glycoprotein, a SARS virus glycoprotein, a West Nile Virus glycoprotein, a herpesvirus simplex glycoprotein, a cytomegalovirus glycoprotein, and combinations thereof.

5. The virosome of claim 1, wherein the at least one adjuvant molecule is selected from: an influenza HA as an adjuvant molecule; a parainfluenza HN as an adjuvant molecule; a Venezuelan equine encephalitis (VEE) glycoprotein adjuvant molecule; a C3d adjuvant molecule; a mannose receptor adjuvant molecule; a membrane-anchored form of a molecule selected from: a mammalian toll-like receptor (TLR) ligand molecule, a MIP-1α molecule, a RANTES MIP-1β molecule, a GM-CSF molecule, a Flt3 ligand molecule, a CD40 ligand molecule, an IL-2 molecule, an IL-10 molecule, an IL-12 molecule, an IL-15 molecule, an IL-18 molecule, and an IL-21 molecule; and combinations thereof.

6. The virosome of claim 5, wherein the membrane-anchored form of a mammalian TLR ligand molecule is selected from: a Prevotella intermedia glycoprotein, a respiratory synctial virus protein F, a fibronectin A domain, fibrinogen, a membrane-anchored form of a bacterial flagellin, a measles virus HA protein, and Pam2Cys lipoprotein/lipopeptide (MALP-2).

7. The virosome of claim 1, wherein at least one viral surface envelope glycoprotein is a chimeric viral surface envelope glycoprotein.

8. The virosome of claim 7, wherein the chimeric viral surface envelope glycoprotein comprises:
at least a portion of the external domain of a viral surface envelope glycoprotein from a first virus, and
at least a portion of one or more of a signal peptide domain, a transmembrane domain, or a C-tail domain of a peptide from a second viral glycoprotein or a cellular protein.

9. A chimeric virosome, comprising:
at least one viral surface envelope glycoprotein expressed on the surface of the virosome; and
at least one adjuvant molecule expressed on the surface of the virosome.

10. The chimeric virosome of claim 9, wherein the at least one adjuvant molecule is from a different biological source than at least one viral surface envelope glycoprotein.

11. The chimeric virosome of claim 9, wherein the viral surface envelope glycoprotein is selected from: a retrovirus glycoprotein, a bunyavirus glycoprotein, a corona virus glycoprotein, an arenavirus glycoprotein, a filovirus glycoprotein, an influenza virus glycoprotein, a paramyxovirus glycoprotein, a rhabdovirus glycoprotein, an alphavirus glycoprotein, a flavivirus glycoprotein, a herpesvirus glycoprotein, a cytomeglavirus glycoprotein, and combinations thereof.

12. The chimeric virosome of claim 11, wherein the retrovirus glycoprotein is selected from: a human immunodeficiency virus (HIV) glycoprotoein, a simian immunodeficiency virus (SIV) glycoprotein, a simian-human immunodeficiency virus (SHIV) glycoprotoein, a feline immunodeficiency virus (FIV) glycoprotoein, a feline leukemia virus glycoprotoein, a bovine immunodeficiency virus glycoprotoein, a bovine leukemia virus glycoprotoein, an equine infectious anemia virus glycoprotoein, a human T-cell leukemia virus glycoprotoein, a mouse mammary tumor virus envelope glycoprotien (MMTV), and combinations thereof.

13. The chimeric virosome of claim 11, wherein the viral surface envelope surface glycoprotein is selected from: a Lassa Fever virus glycoprotein, a RVFV glycoprotein, an Ebola Virus glycoprotein, a VSV glycoprotein, a respiratory syncytial virus glycoprotein, a Hepatitis C Virus clycoprotein, a Herpes Simplex Virus glycoprotein, a cytomegalovirus glycoprotein, and combinations thereof.

14. The chimeric virosome of claim 9, wherein the at least one adjuvant molecule is selected from: an influenza HA adjuvant molecule; a parainfluenza HN adjuvant molecule; a Venezuelan equine encephalitis (VEE) glycoprotein adjuvant molecule; a C3d adjuvant molecule; a mannose receptor adjuvant molecule; a membrane-anchored form of a molecule selected from: a mammalian toll-like receptor (TLR) ligand molecule, a MIP-1α molecule, a RANTES MIP-1β molecule, a GM-CSF molecule, a Flt3 ligand molecule, a CD40 ligand molecule, an IL-2 molecule, an IL-10 molecule, an IL-12 molecule, an IL-15 molecule, an IL-18 molecule, and an IL-21 molecule; and combinations thereof.

15. The chimeric virosome of claim 14, wherein the membrane-anchored form of a mammalian TLR ligand molecule is selected from: a Prevotella intermedia glycoprotein, a respiratory synctial virus protein F, a fibronectin A domain, fibrinogen, a membrane-anchored bacterial flagellin, a measles virus HA protein, and Pam2Cys lipoprotein/lipopeptide (MALP-2).

16. The virosome of claim 9, wherein at least one viral surface envelope glycoprotein is a chimeric viral surface envelope glycoprotein.

17. The virosome of claim 16, wherein the chimeric viral surface envelope glycoprotein comprises:
at least a portion of the external domain of a viral surface envelope glycoprotein from a first virus, and
at least a portion of one or more of a signal peptide domain, a transmembrane domain, or a C-tail domain of a peptide from a second viral glycoprotein or a cellular protein.

18. An immunogenic composition, comprising the chimeric virosome of claim 1 and a pharmacologically acceptable carrier.

19. An immunogenic composition, comprising the chimeric virosome of claim 9 and a pharmacologically acceptable carrier.

20. A method of generating an immunological response in a host, comprising the step of administering an effective amount of the immunogenic composition of claim 18 to the host.

21. A method of generating an immunological response in a host, comprising the step of administering an effective amount of the immunogenic composition of claim 19 to the host.

22. A method of treating a condition, comprising administering to a host in need of treatment an effective amount of the immunogenic composition of claim 18.

23. A method of treating a condition, comprising administering to a host in need of treatment an effective amount of the immunogenic composition of claim 19.

24. A method of determining exposure of a host to a virus, comprising the steps of:
contacting a biological fluid of the host with a chimeric virosome of claim 1 or 9, wherein at least a portion of at least one of the viral surface envelope glycoproteins of the chimeric virosome is of the same virus type to which exposure is being determined, under conditions which are permissive for binding of antibodies in the biological fluid with the virosome; and
detecting binding of antibodies within the biological fluid with the virosome, whereby exposure of the host to the virus is determined by the detection of antibodies bound to the virosome.

25. The method of claim 24, wherein detecting includes the use of a labeled second antibody specific for antibodies in the biological fluid being tested.

26. A method of producing a chimeric virosome in a host comprising:
providing one or more expression vectors, wherein the one or more expression vectors comprise a polynucleotide sequence encoding for at least one viral surface envelope glycoprotein, and a polynucleotide sequence encoding for at least one adjuvant molecule;
introducing the one or more expression vectors into a host cell; and
expressing the at least one viral surface envelope glycoprotein and the at least one adjuvant molecule, whereby the virosome is formed by the cell in a host.

27. The method of claim 26, wherein the one or more vectors are selected from: plasmids, cosmids, viral vectors, chromosomes, minichromosomes, baculovirus vectors, modified vaccinia Ankara (MVA) vectors, plasmid DNA vectors, recombinant poxvirus vectors, bacterial vectors, recombinant baculovirus expression systems (BEVS), recombinant VSV vectors, recombinant alphavirus vectors, recombinant flavivirus vectors, recombinant paramyxovirus vectors, recombinant adenovirus expression systems, recombinant herpesvirus vectors, recombinant DNA expression vectors, and combinations thereof.

28. The method of claim 26, wherein the polynucleotide sequences encoding for the at least one viral surface envelope glycoprotein and the at least one adjuvant molecule are each operably linked to a promoter.

29. The method of claim 28, wherein the promoter is selected from a constitutive promoter and an inducible promoter.

30. The method of claim 28, wherein the promoter is selected from: a baculovirus promoter, a recombinant Modified Vaccinia Ankara (MVA) promoter, a CMV promoter, an EF promoter, an adenovirus promoter, a recombinant VSV promoter, a recombinant alphavirus promoter, a recombinant paramyxovirus promoter, a recombinant adenovirus promoter, a recombinant herpesvirus promoter, a recombinant poxvirus promoter, a recombinant cytomegalovirus promoter, and combinations thereof.

31. The method of claim 26, wherein the expressed chimeric virosome is selected from: human immunodeficiency virus (HIV) virosome, a simian-human immunodeficiency virus (SHIV) virosome, a feline immunodeficiency virus (FIV) virosome, a feline leukemia virus virosome, a bovine immunodeficiency virus virosome, a bovine leukemia virus virosome, a equine infectious anemia virus virosome, a human T-cell leukemia virus virosome, a Rift Valley fever virosome, a Lassa fever virus virosome, an Ebola virus virosome, a corona virus virosome, an Arena virus virosome, a Filovirus virosome, an influenza virus virosome, a paramyxovirus virosome, a rhabdo virus virosome, an alphavirus virosome, and a flavi virus virosome.

32. The method of claim 26, wherein the polynucleotide sequence encoding for at least one viral surface envelope glycoprotein encodes for a chimeric viral surface envelope glycoprotein.

33. A method of immunizing a host comprising:
co-expressing at least one viral surface envelope surface glycoprotein and at least one adjuvant molecule in one or more host cells;
whereby the at least one viral surface envelope glycoprotein and at least one adjuvant molecule assemble to form a virosome.

34. An immunogenic composition, comprising a RVFV glycoprotein virosome and a pharmacologically acceptable carrier.

35. An immunogenic composition, comprising an Ebola glycoprotein virosome and a pharmacologically acceptable carrier.

36. An immunogenic composition, comprising a chimeric HIV Env protein virosome and a pharmacologically acceptable carrier.


SEQUENCE LISTING (11 sequences)

SEQ ID NO: 1 - protein, 847 aa, Human Immunodeficiency Virus
MRVKGIRKNYQHLWRGGTLLLGMLMICSAVEKLWVTVYYGVPVWKEATTTLFCASDAKAYDTEVHNVWATHACVPTDPNPQEIVLENVTENFNMWKNNMVEQMHEDIISLWDQSLKPCVKLTPLCVTLHCTNLKNATNTKSSNWKEMDRGEIKNCSFKVTTSIRNKMQKEYALFYKLDVVPIDNDNTSYKLINCNTSVITQACPKVSFEPIPIHYCAPAGFAILKCNDKKFNGSGPCTNVSTVQCTHGIRPVVSTQLLLNGSLAEEGVVIRSENFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWQEVGKAMYAPPIRGQIRCSSNITGLLLTRDGGKEISNTTEIFRPGGGDMRDNWRSELYKYKVVKIEPLGVAPTKAKRRVVQREKRAVTLGAMFLGFLGAAGSTMGARSLTLTVQARQLLSGIVQQQNNLLRAIEAQQHLLQLTVWGIKQLQARVLAVERYLKDQQLLGIWGCSGKLICTTAVPWNASWSNKSLDQIWNNMTWMEWEREIDNYTNLIYTLIEESQNQQEKNEQELLELDKWASLWNWFDISKWLWYIKIFIMIVGGLVGLRIVFTVLSIVNRVRQGYSPLSFQTRFPAPRGPDRPEGIEEEGGERDRDRSSPLVHGLLALIWDDLRSLCLFSYHRLRDLILIAARIVELLGRRGWEALKYWGNLLQYWIQELKNSAVSLFDAIAIAVAEGTDRIIEVAQRIGRAFLHIPRRIRQGFERALL

SEQ ID NO: 2 - protein, 1067 aa, Rift Valley Fever Virus
MAGIAMTVLPALAVFALAPVVFAEDPHLRNRPGKGHNYIDGMTQEDATCKPVTYAGACSSFDVLLEKGKFPLFQSYAHHRTLLEAVHDTIIAKADPPSCDLQSAHGNPCMKEKLVMKTHCPNDYQSAHYLNNDGKMASVKCPPKYGLTEDCNFCRQMTGASLKKGSYPLQDLFCQSSEDDGSKLKTKMKGVCEVGVQAHKKCDGQLSTAHEVVPFAVFKNSKKVYLDKLDLKTEENLLPDSFVCFEHKGQYKGTMDSGQTKRELKSFDISQCPKIGGHGSKKCTGDAAFCSAYECTAQYANAYCSHANGSGIVQIQVSGVWKKPLCVGYERVVVKRELSAKPIQRVEPCTTCITKCEPHGLVVRSTGFKISSAVACASGVCVTGSQSPSTEITLKYPGISQSSGGDIGVHMAHDDQSVSSKIVAHCPPQDPCLVHGCIVCAHGLINYQCHTALSAFVVVFVFSSIAIICLAVLYRVLKCLKIAPRKVLNPLMWITAFIRWIYKKMVARVAHNINQVNREIGWMEGGQLVLGNPAPIPRHAPIPRYSTYLMLLLIVSYASACSELIQASSRITTCSTEGVNTKCRLSGTALIRAGSVGAEACLMLKGVKEDQTKFLKIKTVSSELSCREGQSYWTGSISPKCLSSRRCHLVGECHVNRCLSWRDNETSAEFSFVGESTTMRENKCFEQCGGWGCGCFNVNPSCLFVHTYLQSVRKEALRVFNCIDWVHKLTLEITDFDGSVSTIDLGASSSRFTNWGSVSLSLDAEGISGSNSFSFIESPSKGYAIVDEPFSEIPRQGFLGEIRCNSESSVLSAHESCLRAPNLISYKPMIDQLECTTNLIDPFVVFERGSLPQTRNDKTFAASKGNRGVQAFSKGSVQADLTLMFDNFEVDFVGAAVSCDAAFLNLTGCYSCNAGARVCLSITSTGTGSLSAHNKDGSLHIVLPSENGTKDQCQILHFTVPEVEEEFMYSCDGDERPLLVKGTLIAIDPFDDRREAGGESTVVNPKSGSWNFFDWFSGLMSWFGGPLKTILLICLYVALSIGLFFLLIYLGRTGLSKMWLAATKKAS

SEQ ID NO: 3 - protein, 853 aa, Human Immunodeficiency Virus
MRVKEIRKNWQHLRGGILLLGMLMICSAAKEKTWVTIYYGVPVWREATTTLFCASDAKAYDTEVHNVWATHACVPTDPNPQEVVLGNVTENFNMWKNNMVDQMHEDIISLWDESLKPCVKLTPLCVTLNCTNLNITKNTTNPTSSSWGMMEKGEIKNCSFYITTSIRNKVKKEYALFNRLDVVPIENTNNTKYRLISCNTSVITQACPKVSFQPIPIHYCVPAGFAMLKCNNKTFNGSGPCTNVSTVQCTHGIRPVVSTQLLLNGSLAEEDIVIRSENFTDNAKTIIVQLNESVVINCTRPNNNTRRRLSIGPGRAFYARRNIIGDIRQAHCNISRAKWNNTLQQIVIKLREKFRNKTIAFNQSSGGDPEIVMHSFNCGGEFFYCNTAQLFNSTWNVTGGTNGTEGNDIITLQCRIKQIINMWQKVGKAMYAPPITGQIRCSSNITGLLLTRDGGNSTETETEIFRPGGGDMRDNWRSELYKYKVVRIEPIGVAPTRAKRRTVQREKRAVGIGAVFLGFLGAAGSTMGAASVTLTVQARLLLSGIVQQQNNLLRAIEAQQHMLQLTVWGIKQLQARVLALERYLRDQQLMGIWGCSGKLICTTSVPWNVSWSNKSVDDIWNNMTWMEWEREIDNYTDYIYDLLEKSQTQQEKNEKELLELDKWASLWNWFDITNWLWYIRLFIMIVGGLIGLRIVFAVLSIVNRVRQGYSPLSFQTLLPASRGPDRPEGTEEEGGERDRDRSGPLVNGFLALFWVDLRNLCLFLYHLLRNLLLIVTRIVELLGRRGWEALKYWWNLLQYWSQELKNSAVSLLNATAIAVAEGTDRVIKIVQRACRAIRNIPTRIRQGLERALL

SEQ ID NO: 4 - protein, 507 aa, Rift Valley Fever Virus
CSELIQASSRITTCSTEGVNTKCRLSGTALIRAGSVGAEACLMLKGVKEDQTKFLKIKTVSSELSCREGQSYWTGSISPKCLSSRRCHLVGECHVNRCLSWRDNETSAEFSFVGESTTMRENKCFEQCGGWGCGCFNVNPSCLFVHTYLQSVRKEALRVFNCIDWVHKLTLEITDFDGSVSTIDLGASSSRFTNWGSVSLSLDAEGISGSNSFSFIESPSKGYAIVDEPFSEIPRQGFLGEIRCNSESSVLSAHESCLRAPNLISYKPMIDQLECTTNLIDPFVVFERGSLPQTRNDKTFAASKGNRGVQAFSKGSVQADLTLMFDNFEVDFVGAAVSCDAAFLNLTGCYSCNAGARVCLSITSTGTGSLSAHNKDGSLHIVLPSENGTKDQCQILHFTVPEVEEEFMYSCDGDERPLLVKGTLIAIDPFDDRREAGGESTVVNPKSGSWNFFDWFSGLMSWFGGPLKTILLICLYVALSIGLFFLLIYLGRTGLSKMWLAATKKAS

SEQ ID NO: 5 - protein, 527 aa, Rift Valley Fever Virus
EDPHLRNRPGKGHNYIDGMTQEDATCKPVTYAGACSSFDVLLEKGKFPLFQSYAHHRTLLEAVHDTIIAKADPPSCDLQSAHGNPCMKEKLVMKTHCPNDYQSAHYLNNDGKMASVKCPPKYGLTEDCNFCRQMTGASLKKGSYPLQDLFCQSSEDDGSKLKTKMKGVCEVGVQAHKKCDGQLSTAHEVVPFAVFKNSKKVYLDKLDLKTEENLLPDSFVCFEHKGQYKGTMDSGQTKRELKSFDISQCPKIGGHGSKKCTGDAAFCSAYECTAQYANAYCSHANGSGIVQIQVSGVWKKPLCVGYERVVVKRELSAKPIQRVEPCTTCITKCEPHGLVVRSTGFKISSAVACASGVCVTGSQSPSTEITLKYPGISQSSGGDIGVHMAHDDQSVSSKIVAHCPPQDPCLVHGCIVCAHGLINYQCHTALSAFVVVFVFSSIAIICLAVLYRVLKCLKIAPRKVLNPLMWITAFIRWIYKKMVARVAHNINQVNREIGWMEGGQLVLGNPAPIPRHAPIPRYSTYLM

SEQ ID NO: 6 - protein, 26 aa, Simian Immunodeficiency Virus
YIQYGVYIVVGVILLRIVIYIVQMLA

SEQ ID NO: 7 - protein, 232 aa, Mus Musculus
MTVLAPAWSPNSSLLLLLLLLSPCLRGTPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTCTQLLALKPCIGKACQNFSRCLEVQCQPDSSTLLPPRSPIALEATELPEPRPRQLLLLLLLLLPLTLVLLAAAWGLRWQRARRRGELHPGVPLPSHP

SEQ ID NO: 8 - protein, 164 aa, Simian Immunodeficiency Virus
KLRQGYRPVFSSPPSYFQQTHIQQDPALPTREGKERDGGEGGGNSSWPWQIEYIHFLIRQLIRLLTWLFSNCRTLLSRVYQILQPILQRLSATLQRIREVLRTELTYLQYGWSYFHEAVQAVWRSATETLAGAWGDLWETLRRGGRWILAIPRRIRQGLELTLL

SEQ ID NO: 9 - protein, 23 aa, Homo Sapiens
MDAMKRGLCCVLLLCGAVFVSAR

SEQ ID NO: 10 - protein, 40 aa, Homo Sapiens
LAVSTISSFFPPGYQVSFCLVMVLLFAVDTGLYFSVKTNI

SEQ ID NO: 11 - protein, 300 aa, Mus Musculus
TVIAVHYLDQTEQWEKFGIEKRQEALELIKKGYTQQLAFKQPSSAYAAFNNRPPSTWLTAYVVKVFSLAANLIAIDSHVLCGAVKWLILEKQKPDGVFQEDGPVIHQEMIGGFRNAKEADVSLTAFVLIALQEARDICEGQVNSLPGSINKAGEYIEASYMNLQRPYTVAIAGYALALMNKLEEPYLGKFLNTAKDRNRWEEPDQQLYNVEATSYALLALLLLKDFDSVPPVVRWLNEQRYYGGGYGSTQATFMVFQALAQYQTDVPDHKDLNMDVSFHLPSRSSATTFRLLWENGNLLR